(19) United States
(12) Patent Application Publication
(10) Pub. No.: US 2023/0277118 A1
(43) Pub. Date: Sep. 7, 2023

(54) METHODS AND SYSTEMS FOR DETECTING AND ASSESSING COGNITIVE IMPAIRMENT

(71) Applicant: SPARK NEURO INC., New York, NY (US)

(72) Inventors: Ché Lucero, Albuquerque, NM (US); Geoffrey Brookshire, Mill Valley, CA (US); Keith J. Yoder, Chicago, IL (US); Yunan Wu, Pittsburgh, PA (US); Colin Quirk, Chicago, IL (US); Spencer Gerrol, West Hartford, CT (US)

(21) Appl. No.: 18/105,384

(22) Filed: Feb. 3, 2023

Related U.S. Application Data
(60) Provisional application No. 63/306,914, filed on Feb. 4, 2022.
(60) Provisional application No. 63/306,915, filed on Feb. 4, 2022.

(51) Int. Cl. A61B 5/00

(52) U.S. Cl. CPC A61B 5/4088

(57) ABSTRACT

The present invention provides systems and methods for assessing Alzheimer’s disease using Fractal Dimension Distributions (FDD).

### TECHNICAL FIELD

The present disclosure relates to methods and systems using neural data to assess neural cognition and pathologies.

### BACKGROUND

Neurological disorders are the leading cause of disability-adjusted life-years (sum of years of life lost and years lived with a disability) and the second leading cause of deaths. Alzheimer’s disease (AD) and related dementias or (ADRD), Parkinson’s disease and motor neuron diseases including amyotrophic lateral sclerosis, spinal muscular atrophy, hereditary spastic paraplegia, primary lateral sclerosis, progressive muscular atrophy, and pseudobulbar palsy collectively affected 4.7 to 6.0 million individuals in the U.S. between 2016-2017. By 2060, the prevalence ADRDs is expected to double. Accordingly, there has been intense interest in developing methods and systems that can accurately diagnose neurological disorders, assess their progression, and provide insight into appropriate treatment options.

Healthy brains sustain complex activity that shows similarity across different time scales, analogous to the way fractals display similarity at different spatial scales. A well-established characteristic of Alzheimer’s disease is a “slowing” of neural activity. For example, an Alzheimer patient’s dominant Alpha rhythm may gradually downshift (e.g., 12 Hz to 11 Hz), as their disease progresses. Literature has considered aspects of spectral power (e.g., peak alpha frequency, power ratio between delta and alpha activity etc.) within individual frequency bands when discriminating healthy verses Alzheimer’s Disease patients using magnetoencephalography (MEG) or electroencephalogram (EEG) recordings.

However, current techniques for assessing neurological disorders, including AD, do not consider assessing neural complexity using an analysis that assesses fractal-character activity within different frequency bands. Rather, current methods may use whole-recording wideband data (e.g., from an EEG or MEG) to examine a single fractal dimension score for a timeseries, or use within-band analyses that do not consider fractal-character activity. Thus, such methods lack the necessary detail needed to reliably discriminate between, for example, patients with AD from those without AD, especially within mild cognitive impairment (MCI) and dementia patient groups. Discriminating AD from non-AD cases in cognitively impaired or demented patients is a much more challenging task compared to discriminating healthy versus AD patients, and attempts of the former appear rarely in the academic literature.

### SUMMARY

The present invention includes systems for assessing a subject to provide a prediction of whether a subject has or will develop Alzheimer’s disease (AD), i.e., the subject’s AD status. Across a large number of resting-state electroencephalography (EEG) studies, dementia is associated with changes to the power spectrum and fractal dimension. The present invention employs a novel method to assess changes in fractal dimension values over time both wideband and within frequency bands. This method, called Fractal Dimension Distributions (FDD), combines spectral and complexity information. The method examines EEG data recorded from patients to assess and discriminate Alzheimer’s and/or other cognitive impairments.

Surprisingly, the present methods using FDD reveal larger group differences in patients experiencing subjective cognitive impairment (SCI) or dementia detectable at greater numbers of EEG recording sites than using methods without FDD. Moreover, linear models using FDD features have lower AIC and higher R² than models using standard full time-course measures of fractal dimension. Methods using the presently disclosed FDD metrics also outperform full time-course metrics when comparing patients with SCI to dementia patients diagnosed with Alzheimer’s disease (AD). FDD offers unique information beyond traditional full time-course fractal analyses, and can be used to identify dementia and to distinguish AD dementia from non-AD dementia.

In certain aspects, a system of the invention includes a central processing unit (CPU); and storage coupled to said CPU for storing instructions that when executed by the CPU cause the CPU to: accept as an input, neural data from a subject, recorded using EEG sensors; transform the neural data into a time series; bandpass filter the time series to produce a plurality of different frequency band time series; repeatedly advance a sliding-window along each frequency band time series and obtain at least one fractal measurement to produce at least one measure time series for each frequency band time series; extract measures from each measure time series; combine the extracted measure and analyze the combined measures using a machine learning system trained to correlate features in the combined measures with Alzheimer’s disease to produce at least one fractal dimension distribution (FDD) score; and provide an output of the subject’s AD status based on the FDD score.

In certain aspects, producing the FDD score includes determining summary statistics for the distribution of the fractal values. Summary statistics may be, for example, one or more of a standard deviation of the fractal values, mean of the fractal values, skewness of the fractal values, and kurtosis of the fractal values.

In certain aspects, the extracted measures include one or more of: frequency and/or time frequency measures; oscillatory measures; amplitude modulation measures; spectral connectivity measures; network analysis measures; chaos measures; complexity measures; and entropy measures.

In certain systems, the output provides a continuous score indicating the probability that a subject has Alzheimer’s disease. The systems may allocate the continuous score into one of a plurality of bins, wherein each bin corresponds to a subject’s risk of developing Alzheimer’s disease. When the probability drops below a threshold, the output may provide an indication that the subject does not have Alzheimer’s disease.

The systems may further include in an output an identification of the extracted measures analyzed by the machine learning system and/or the FDD scores use to provide the output. The extracted measures provided in the output may be identified using a pictorial representation of the analyzed measure.

In certain aspects, the systems of the invention may record neural data from a subject over at least one period of time during which the subject is at rest either with their eyes open, their eyes closed, or both. In certain aspects, the neural data is collected from the subject from at least two periods of time. The systems may provide an output of the subject’s AD status after every period of time during which neural data is recorded from the subject. In certain aspects, results from each output are combined to produce a longitudinal assessment of the subject’s AD status.

In certain aspects, the recorded neural data includes one or more annotations identifying one or more of the subject’s age, sex, medical history, results from one or more biomolecular assay, and/or subjective cognitive assessment. These annotations may be provided to the machine learning system for analysis with the combined extracted measures.

The present invention also provides methods for assessing an AD status of a subject to provide a prediction of whether a subject has or will develop Alzheimer’s disease. In certain aspects, a method for assessing the AD status of a subject includes: recording using EEG sensors neural data from a subject using EEG sensors; transforming the neural data into a time series; filtering the time series to produce a plurality of different frequency band time series; producing at least one measure time series for each frequency band time series by repeatedly advancing a sliding-window along each frequency band time series and obtaining at least one fractal measurement; extracting measures from each measure time series; combining the extracted measure and analyzing the combined measures using a machine learning system trained to correlate features in the combined measures with Alzheimer’s disease to produce at least one fractal dimension distribution (FDD) score; and providing an output of the subject’s AD status based on the FDD score.

In certain methods, producing the FDD score comprises determining summary statistics for the distribution of the fractal measurements. The summary statistics may comprise one or more of a standard deviation of the fractal measurements, and skewness of the fractal measurements, kurtosis of the fractal measurements.

In certain methods, the extracted measures comprise one or more of: frequency and/or time frequency measures; oscillatory measures; amplitude modulation measures; spectral connectivity measures; network analysis measures; chaos measures; complexity measures; and entropy measures.

In methods of the invention, the output may include a continuous score indicating the probability that a subject has Alzheimer’s disease. Such methods may further include allocating the continuous score into one of a plurality of bins, wherein each bin corresponds to a subject’s risk of developing Alzheimer’s disease. In certain methods, when the probability drops below a threshold, the output provides an indication that the subject does not have Alzheimer’s disease.

In certain aspects, the output includes an identification of the extracted measures analyzed by the machine learning system and/or the FDD scores use to provide the output. The extracted measures in the output may be identified using a pictorial representation of the analyzed measure.

In certain methods, the neural data is recorded from the subject over at least one period of time during which the subject is at rest. In some methods, the neural data is recorded from the subject during at least two periods of time. The methods may also include providing an output of the subject’s AD status after every period of time during which neural data is recorded from the subject. Such methods include combining the results from each output to produce a longitudinal assessment of the subject’s AD status.

Methods of the invention may further include annotating the recorded neural data with one or more annotations identifying one or more of the subject’s age, sex, medical history, results from one or more biomolecular assay, and/or subjective cognitive assessment. The annotations may be provided to the machine learning system for analysis with the combined extracted measures.

### BRIEF DESCRIPTION OF DRAWINGS

FIG. 1 shows a workflow in accordance with the invention.

FIG. 2 shows a schematic of producing frequency band time series.

FIG. 3 shows the use of a sliding-window to produce a measure time series.

FIG. 4 shows a schematic of producing FDD scores using measure time series.

FIG. 5 shows an exemplary output of a system of the invention.

FIG. 6 shows a map of EEG sensors.

FIG. 7 shows an exemplary system of the invention.

FIG. 8 shows an exemplary report section.

FIG. 9 shows an exemplary report section.

FIG. 10 shows instructions displayed by certain systems of the invention.

FIG. 11 provides scalp plots showing differences (Dementia - SCI) calculated using threshold-free cluster enhancement (TFCE) for Higuchi fractal dimension (left) or Katz fractal dimension (right). Electrodes with significant differences (TFCE FWEp < 0.05) are marked with white circles.

FIG. 12, absolute TFCE-t statistic averaged across channels for group differences (Dem -SCI) in each frequency band using Higuchi (A) or Katz FD (C). Difference between FDD metrics and full time-course FD in each frequency band (B and D). Box outlines show the interquartile range, central line shows the median, and whiskers extend to minimum and maximum value.

FIG. 13 provides the resulting scalp plots that show differences (AD - SCI) calculated using threshold-free cluster enhancement (TFCE) for Higuchi fractal dimension (left) or Katz fractal dimension (right).

FIG. 14 provides the average channel absolute TFCE-t statistic for AD-SCI in each frequency band using Higuchi (A) or Katz (C). Difference between FDD metrics and full time-course FD in each frequency band (B and D). Whiskers extend to minimum and maximum value.

FIG. 15 provides channels with non-zero coefficients in the regularized logistic regression using full time-course FD (left) or FDD (center and right) features calculated using Higuchi (top) or Katz (bottom) methods.

FIG. 16 shows correlation of the fractal scores obtained from EEG data across channels in the fractal dimension.

FIG. 17 shows correlation of the fractal scores obtained from EEG data across channels in the mean fractal dimension.

FIG. 18 shows correlation of the fractal scores obtained from EEG data across channels in the standard deviation fractal dimension.

### DETAILED DESCRIPTION

The present invention includes systems and methods for assessing the cognitive function in a subject using the novel Fractional Dimensional Distributions (FDD) technique. FDD provides a new class of measures that have proven effective in detecting the presence of neurodegeneration caused by Alzheimer’s disease (AD) in subject using only non-invasive brain imaging techniques. The present Inventors developed the FDD-based methods and systems of the invention on the insight that AD-afflicted brains have trouble sustaining complex activity, and that activity in different oscillatory bands is differentially impacted by the progression of Alzheimer’s Disease. The presently disclosed FDD techniques measure stability of the complexity of a brain’s activity within particular oscillatory bands. It therefore makes a surprising improvement on both approaches that summarize brain activity complexity without regard to the moment-to-moment changes in a brain’s ability to sustain that complexity, as well as naive approaches that consider only the spectral power of neural activity in different oscillatory bands without regard to the complexity of that oscillatory activity.

Neurotypical brains sustain complex activity that shows similarity across different time scales, which is analogous to the way fractals display similarity at different spatial scales. Signal processing measures such as Katz Fractal Dimension and Higuchi Fractal Dimension have been developed to characterize that type of activity within time-varying signals. This family of signal processing techniques have been applied to neuroimaging recordings to characterize the complexity of brain activity. When applied to EEG recordings, the resulting fractal measure values have been shown to be helpful in classifying healthy patients versus Alzheimer’s Disease patients. A general approach is to compute the fractal measure on an entire EEG recording at each sensor, yielding 1 value per EEG sensor per subject (e.g., 19 HFD values computed from a 5-minute EEG recording using a 19-channel cap), a whole-recording wideband technique.

A well-established characteristic of Alzheimer’s disease is a “slowing” of neural activity. For example, an Alzheimer patient’s dominant Alpha rhythm may gradually downshift (e.g., from 12 Hz to 11 Hz) as their disease progresses. This slowing is also evident when looking across oscillatory bands, where the predominance of spectral power shifts to lower frequencies. As the disease progresses activity in the delta (1 Hz to 4 Hz) and theta (4 Hz to 8 Hz) bands increases, accompanied by attenuation in the alpha (8 Hz to 12 Hz), beta (13 Hz to 30 Hz), and gamma (30 Hz and greater) bands. Thus, prior methods for assessing AD status considered aspects of spectral power (e.g., peak alpha frequency, power ratio between delta and alpha activity, etc.) within different bands when discriminating healthy versus Alzheimer’s Disease patients using magnetic encephalography (MEG) or EEG.

In certain aspects, the presently disclosed systems and methods using FDD separate a time series of neural activity (e.g., from an EEG recording) into frequency-banded time series, to measure stability of the complexity of a brain’s activity within different, particular oscillatory bands. Thus, the systems and methods of the invention improve upon prior approaches that summarize brain activity complexity without considering to the moment-to-moment changes in a brain’s ability to sustain that complexity, or naive approaches that consider only the spectral power of neural activity in different oscillatory bands without considering to the complexity of that oscillatory activity.

Although the methods and systems using FDD described herein are preferably used with neural data provided by EEG recordings, the FDD may also be computed using timeseries of neural activity such as that derived from MEG, fNIRS, or MRI.

The present Inventors have discovered that, surprisingly, by using the systems and methods of the disclosure, the AD status of a subject may be determined with an accuracy of 96%, sensitivity of 90%, and specificity of 99%. As described in the Examples herein, the presently disclosed systems and methods surpass conventional methods, which do not employ FDD.

In certain aspects, the systems and methods of the invention employ a classifier that includes one or more machine learning models.

Machine learning (ML) is branch of computer science in which machine-based approaches are used to make predictions. (Bera et al., Nat Rev Clin Oncol., 16(11):703-715 (2019)). ML-based approaches generally require training a system, such as a familiarity classifier, by providing it with annotated training data. The system learns from data fed into it, to make and/or refine predictions. Id. Machine learning differs from, rule-based or statistics-based program models. (Rajkomar et al., N Engl J Med, 380:1347-58 (2019)). Rule-based program models rely on explicit rules, relationships, and correlations. Id.

In contrast, an ML model learns from examples fed into it, and creates new models and routines based on acquired information. Id. Thus, an ML model may create new correlations, relationships, routines or processes never contemplated by a human. A subset of ML is deep learning (DL). (Bera et al. (2019)). DL uses artificial neural networks. A DL network may include layers of artificial neural networks. Id. These layers may include an input layer, an output layer, and multiple hidden layers. Id. DL is able to learn and form relationships that exceed the capabilities of humans. (Rajkomar et al. (2019)).

By combining the ability of ML, including DL, to develop novel routines, correlations, relationships and processes amongst complex data sets, such as the EEG recordings used in the systems and methods of the inventions, the classifiers described herein can provide accurate and insightful assessments neural data to provide an accurate assessment of a subject’s AD status.

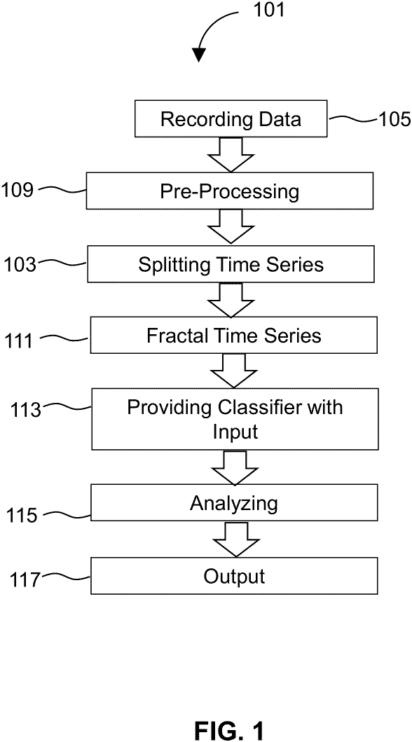

FIG. 1 provides a generalized workflow 101 used in connection with the systems and methods of the invention. As shown, the workflow 101 requires recording neural data from a subject 105, preferably using EEG electrodes placed on the subject’s scalp.

In certain methods and systems, the EEG measurements obtained from the subject undergo a pre-processing step 109. Methods and systems of the invention may employ a number of pre-processing techniques, including but not limited to, amplifying EEG recorded voltages, converting analogue EEG signals into digital signals, filtered, bandpass filtered, baseline corrected, referenced, referenced, normalized. This pre-processing step 109 converts the raw electrical voltage potentials from the EEG neural recording into a time series. Preprocessing may include a variety of steps, analytical components, hardware modules and/or software operations.

In preferred systems and methods, preprocessing 109 includes removing artifacts, such as electromyographic artifacts those caused by a subject’s behaviors (e.g., muscle contractions and blinking or moving their eyes), environmental artifacts (e.g., a door closing or leaky electronics use nearby during recording), and other artifacts or “jumps” produced during use of the high-impedance electrodes of an EEG. Removing artifacts may be accomplished, for example, by using an independent component analysis algorithm.

In preferred systems and methods of the invention, EEG data is preprocessed using an automated pipeline. The pipeline may interpolate any brief jump artifacts present, which may be identified by, for example a sample-to-sample change of greater than about 8 to about 10 standard deviations with elevated values lasting less than 100 ms. The data may be band-pass filtered, for example, with 0.01 Hz and 50 Hz cutoff values. In certain aspects, the data is re-referenced to the common average. Methods and systems may use template-matching to identify artifacts in the raw EEG data. Aspects of the EEG data may be compared with templates characteristic of one or more EEG artifacts. If the compared aspects of the raw EEG match such a template, an artifact may be identified in the raw EEG data. Certain methods and systems use Independent Component Analysis (ICA) in which components from the raw EEG data are extracted and compared to the same components in EEG artifact templates. In certain aspects, the ICA extracts at least 1, 2, 3, 4, 5, 6, 7, 8, 9, 10, 11, 12, 13, 14, 15, 16, 17, 18, 19, or 20 components from the raw EEG data. Certain methods and systems may use an automated Independent Component Analysis (ICA) method that uses template-matching to identify and remove ICA components that captured EEG artifactual activity created, for example, by a subject’s eye saccades and eye blinks. Templates, e.g., indicative of eye movement or intersystem artifacts, may include a number of components identifiable by ICA extraction and analysis.

In certain aspects, artifacts may be removed and replaced with a linear interpolation between the nearest samples that were not contaminated by artifacts. In preferred aspects, the linear interpolation is computed separately for each channel.

The time series data can further be further transformed during the pre-processing step, for example by a Fourier transform or fast Fourier transform, into a spectral and/or time-frequency domain.

In FDD, the pre-processing step 109 includes decomposing the neural data from a time series to produce a plurality of frequency band time series.

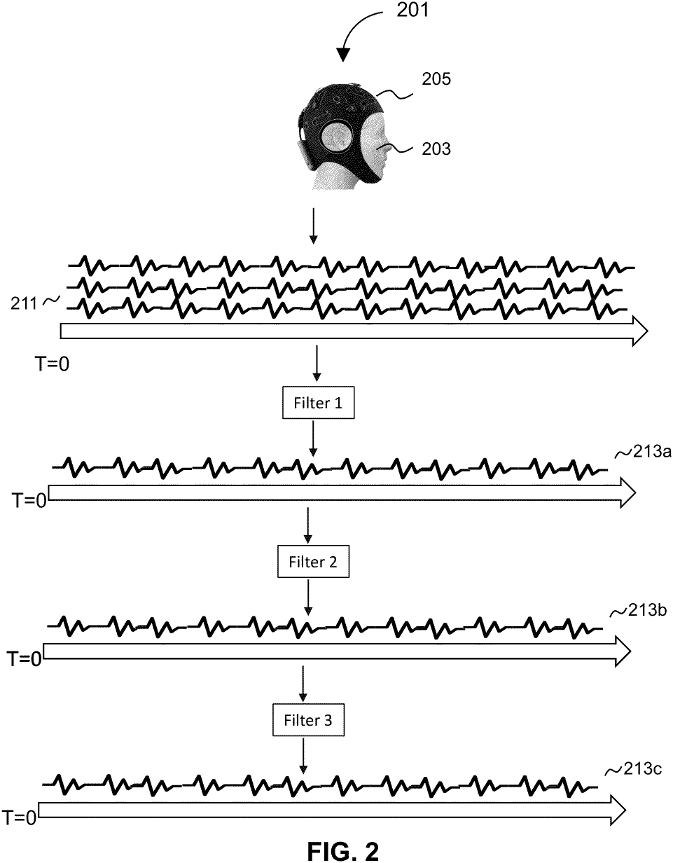

FIG. 2 provides a schematic 201 of producing frequency band time series. As shown in FIG. 2, a cap of EEG sensors 205 is placed on a subject 203 and neural data recorded, preferably, while the subject 203 is at rest. Systems and methods of the invention may use this recorded neural data to produce a time series 211. The time series 211 includes whole-recording broadband neural data.

This whole-recording data is filtered using a series of bandpass filters. Every filter applied produces a specific frequency band time series 213a, 213b, 213c. In certain aspects, the neural data is decomposed to provide neural data frequency band time series in, for example, one or more of the delta band (0.5-4 Hz), the theta band (4-8 Hz), the alpha band (8-12 Hz), SMR (12-15 Hz), the beta band (12-30 Hz) and gamma band (30 Hz and up). In certain aspects, band frequency time series are created for neural sub-bands, e.g., betal/low beta (13-17 Hz) and beta2/high beta (17-30 Hz). The present Inventors made the surprising discovery that splitting the time series into discrete frequency band time series provided deeper, more accurate results as compared to using single channels or whole-recordings.

The pre-processing step 109 may further include normalizing neural data at one or more specific frequency to that of another frequency. For example, in preferred methods and systems, neural data at 6 Hz is used to normalize the neural data in the frequency band time series.

Returning to the method described in FIG. 1, after pre-processing 109 and splitting/filtering 103 the whole-recoding time series into frequency band time series. The systems and methods of the invention using FDD produce 111 at least one fractal time series from each frequency band time series.

In certain aspects, producing the at least one fractal time series 111 includes assigning windows or bins to each frequency band time series. Typically, window or bin applies to a subset of a set of data with the implication that, for linear (e.g., over time) data, values of those data will be put into sets, dubbed windows or bins (that may or may not overlap), where those sets are suitable to an analysis as inputs for the classifiers disclosed herein.

Preferably, the window size is tunable. An exemplary system of the invention may use a window with 1 second of data (500 samples). After computing the measures within a given window, the system advances the window. The amount of time or samples the window is advanced is tunable and is 100 ms (50 samples) in an exemplary system. The system then computes the measures again in the new window. This process continues until the entire timeseries has been processed through the sliding window approach, yielding measure/fractal timeseries.

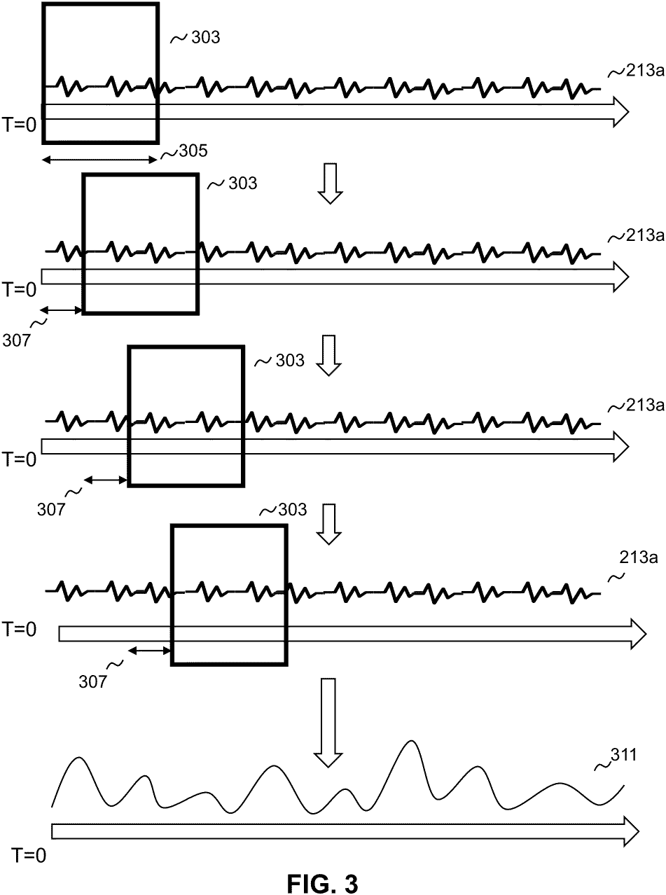

FIG. 3 provides a schematic for producing a fractal time series. In FIG. 3, a sliding-window 303 is applied to a frequency band time series 213a. The sliding-window includes a size 305 in time. For example, in preferred aspects, a window has a size of around 1 to 100 second. In certain aspects, the window has a size of around 1-2 seconds. The window 303 also has a step/stride 307. As shown in FIG. 3, the step/stride 307 is the amount of time along the time series that the window is advanced.

In preferred aspects, at each step/stride at least one fractal measure is obtained from the time series. In certain aspects, a plurality of fractal measures is obtained from each time series at each step/stride. The value of the fractal measure(s) obtained at each step is recorded as the window slides across the frequency band time series. The saved values are a new timeseries of fractal values that captures the stability of the complexity of the brain’s activity within a particular oscillatory band. As a result, the fractal measures for each frequency band time series are used to create a measure/fractal time series 311 from each frequency band time series.

By way of illustration, in an exemplary method of the invention an EEG recording is preprocessed (e.g., removal of EMG, eye blink, and eye movement artifacts using ICA, re-referencing the EEG recording to a common average, etc.). Optionally, the EEG data may be submitted to a source-localization algorithm such as eLORETA, yielding neural activity timeseries attributable to neuroanatomical locations, rather than sensor locations on the scalp. The recording is filtered into different frequency bands (e.g., delta, theta, alpha, beta1, beta2, gamma), yielding as many timeseries as band filters. Within each filtered timeseries the data is transformed into a timeseries of fractal values using the sliding window technique described in FIG. 3. The window is defined by the amount of neuroimaging timeseries data it captures (e.g., two seconds of timeseries) and a step size that defines the amount of time the window is advanced along the timeseries at each step (e.g., 50 ms).

The window initially captures the first two seconds of a timeseries. A fractal measure such as the Higuchi fractal dimension (HFD) is computed from the data within the window, and the resulting HFD value is saved. Then the window is advanced by the step size along the filtered neuroimaging timeseries, and a new HFD value is computed and saved. This is repeated along the filtered timeseries until the final sample has been included once in a fractal computation. The saved values are a new timeseries of fractal values that captures the stability of the complexity of the brain’s activity within a particular oscillatory band.

In certain aspects, in each window slid along a frequency band time series, more than one fractal measure is obtained at each step. In certain aspects, different fractal measures are used to produce different fractal/measure time series from each frequency band time series. In certain aspects, a combination of fractal measures are combined to produce a fractal/measure time series from each frequency band time series.

Exemplary fractal measures used in the methods and systems of the invention include one or more complexity measures such as the Higuchi fractal dimension (HFD) and the Katz fractal dimension (KFD). The HFD is nonlinear measure for how much variation there is in the signal. When the signal is rhythmic with repeating patterns, HFD is low. However, if the signal is more complex with more variations and less repetition, HFD is high. Similar to HFD, KFD also measures the self-similarity of a signal. However, HFD does so by subsampling the signal and analyzing the signal similarity within each subsample. KFD involves calculating the average distance between successive points in a signal.

In certain aspects, the methods and systems of the invention, the KFD is calculated. Alternatively or additionally, the HFD is calculated. HFD starts by subsampling a time-series across progressive smaller time scales. Thus, HFD depends on both the number of sample points (N) and the parameter kₘₐₓ, which sets the upper limit on the number of time intervals (Higuchi, 1988). In certain aspects, the HFD is calculated using kₘₐₓ = 6 as described in Accardo et al., (1997), incorporated herein by reference. Other approaches suggest calculating HFD across multiple kₘₐₓ values and identifying the value of kₘₐₓ at which HFD plateaus (Doyle et al., 2004; Wajnsztejn et al., 2016). However, HFD is not guaranteed to plateau. Accordingly, in preferred aspects, optimal kₘₐₓ is determined on the length of the time-series of the full time-course or 1 s windows as decbribed by Wanliss & Wanliss, (2022), which is incorporated herein by reference.

In certain methods, the calculated KFD and/or HFD is determined using a software package, such as the AntroPy package (version 0.1.4). In certain methods and systems, the HFD and/or KFD is extracted from each moving window (e.g., windows of 1 second) with an overlap between windows (e.g., a 0.5 second overlap), and the distribution of HFD and/or KFD values is summarized across windows using the mean and standard deviation to provide fractal dimension distributions.

In certain aspects, the methods and systems of the invention obtain one or more of Lyapunov Exponent (LE), Hjorth mobility (HM), Hjorth complexity (HC), sample entropy (SaE), spectral entropy (SpE), approximate entropy, multiscale entropy (MSE), permutation entropy (PE) and Hurst exponent (HE) from each window to produce a fractal/measure time series.

Lyapunov Exponents measure growth rates of generic perturbations of a dynamical system.

Entropy is a concept in information theory that quantifies how much information is in a probabilistic event. The more predictable an event is (extremely high or extremely low probabilities), the less information there is and therefore the lower the entropy values. SpE, also known as the Shannon Entropy of the spectrum that measures how flat the spectrum is: the higher the value, the flatter the power spectrum (which means there are less peaks in different frequency components). Approximate entropy is a measure to quantify the amount of regularity and predictability in a signal, similar to a complexity measure.

SaE is an improvement of approximate entropy with decreased bias and more accurate estimate of the complexity of a signal. Similar to HFD, it also subsamples the signal and calculates distance metrics for the subsamples. Multiscale entropy is an extension of sample entropy (or approximate entropy) where instead of just calculating sample entropy based on single samples in the EEG data, it also time windows with varying window lengths and calculates the sample entropy of those time windows. MSE potentially contains more information than just the sample entropy: it also measures self-similarity or complexity of the signals for longer time ranges.

Permutation Entropy subsamples a signal, orders the subsample by magnitude and summarizes the ordering information within the entire signal, which quantifies the pattern of change in the signal.

In certain aspects, the system performs a frequency decomposition via a Hanning-windowed Fourier transform on the fractal/measure timeseries to characterize oscillations in how a given measure at a given location changed over the recording period. For instance, the system can compute the strength of delta oscillations in the beta-band filtered Katz Fractal Dimension time series that was computed from a timeseries source localized to the left hippocampus.

In certain aspects, features/measures extracted from the frequency band time series and/or the fractal/measure time series include one or more of relative and absolute delta, theta, alpha, beta and gamma sub-bands, amplitude modulation measures, percentage modulation energy, the band ratios (e.g., theta/beta ratio, delta/alpha ratio, (theta+delta)/(alpha+beta) ratio), relative power, and/or the peak or mode frequency in the power spectral density distribution, the median frequency in the power spectral density, power spectral density mean, average, frequency, standard deviation, skewness, and/or kurtosis.

In certain aspects, pre-processing includes identifying a set or subset of EEG electrodes or sensors from which EEG signals are obtained during recording. EEG electrodes or sensors are arrayed on a cap, and each sensor/electrode provides an identifiable channel that records neural data from a subject. In certain aspects, the neural data recorded from a subset of sensors can be retained, grouped, or discarded.

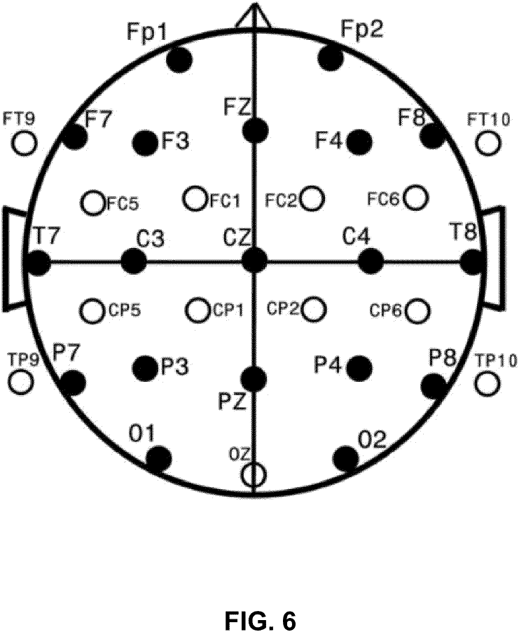

FIG. 6 provides a schematic of a 10-10 system multi-channel EEG array positioned on the head of a subject. Although this schematic shows a 10-10 multi-channel EEG, other EEG systems, such as 10-20 and 10-5 systems, can be used with the systems and methods of the invention.

As shown in FIG. 6, the 10-10 system includes 32 electrodes that are positioned on the scalp of a subject. Each electrode is identified according to the standard 10-10 system nomenclature. In certain aspects, when using a 32 electrode array neural data recorded from a subset of sensors includes data recorded from a subset that includes 1 electrode, or 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 electrodes of the array.

In certain aspects, the pre-processing step 109 includes referencing the recorded neural data by one or more EEG sensor to that recorded by one or more other sensors of the array. In certain aspects, the neural data recorded by a sensor or subset of sensors is referenced to a single other sensor of the array. In certain aspects, the single other sensor of the array is a vertex sensor, such as CZ on a 10-10 array. For example, in certain aspects, neural data recorded from a subset of about 8 bilateral and sagittal midline sensors is referenced to a vertex sensor, such as the Cz on a 10-10 array. After referencing, the data recorded from the referenced sensor(s) may be discarded and not used as an input for the familiarity classifier.

In certain methods and systems of the disclosure neural data recorded from one or more sensors is referenced to that recorded by a plurality of sensors or all sensors of the EEG array. In certain aspects, this includes averaging the data recorded by the plurality of sensors, and referencing the recorded data to the averaged data. Methods and systems of the invention may further include referencing recorded neural data from one or more sensors to that of a single sensor and subsequently re-referencing to a plurality of sensors and/or the entire sensor array.

In certain aspects, preprocessing may include transforming the recorded neural data to produce the whole-recording neural data. In certain aspects, raw neural data (e.g., EEG recordings) is collected from the sensors without any transformations to infer where in the brain signals were recorded from.

In certain aspects, surface Laplacian is used on the recorded neural data. Surface Laplacian is commonly referred to as the current source density (CSD) or scalp current density (SCD). It uses a spatial filter to filter out low spatial frequency data and thus reduce the problem of volume conduction. Surface Laplacian can help increase the accuracy of functional/spectral connectivity results.

In certain aspects, recorded neural data is transformed from original sensor data using algorithms such as eLORETA (exact Low Resolution Electromagnetic Tomogrpahy) to infer which specific brain regions the signals are coming from. Source space data are neurologically meaningful because it can directly observe the origin of a signal and understand the function or impairment of a particular brain structure.

In certain aspects, the pre-processing step 109 includes annotating data provided as an input to a classifier used in the systems and methods of the invention, including when used as training data. Annotation can be performed automatically by the systems of the disclosure and/or by human action or direction. Annotations can be used as features by the classifier to discern or create correlations regarding AD status and recorded neural data using the FDD technique.

Annotations may additionally or alternatively include, for example, the date, time, or location of an AD assessment. Annotations may include information derived or obtained from Electronic Medical Records (EMR) or clinical trial records. In preferred aspects, annotations may include the results of a subjective cognition test and/or determined cognitive impairment (e.g., mild cognitive impairment (MCI) or severe cognitive impairment (SCI).

After the pre-processing step 109, splitting the time series 103 into frequency band time series, the resulting fractal/measure time series have been transformed from raw neural signals into a format that the classifier can use to provide a predication regarding a subject’s AD status.

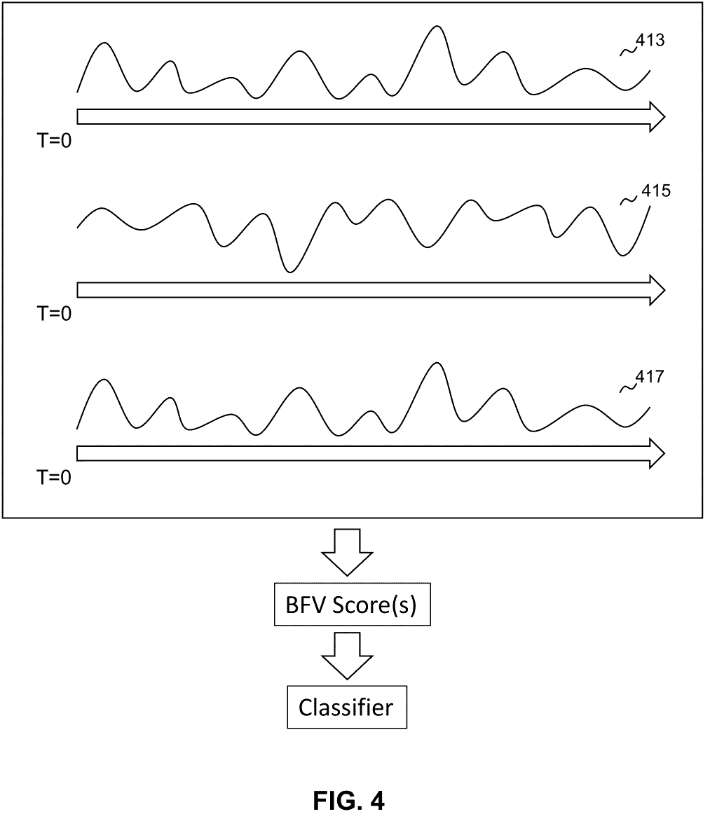

As shown in FIG. 4, fractal/measure time series 413,415,417 are combined and used to determine one or more FDD scores. FDD scores are summary statistics describing the distributions of the fractal time series values. Exemplary FDD scores include, for example, standard deviation, mean, skewness, and kurtosis of the fractal timeseries.

As also shown in FIG. 4, the FDD scores are provided to a classifier is trained to extract measures or features from the FDD scores and correlate them with an AD status in a subject. The FDD scores include features and measures that carry information about the disease status of the patient and even the pathology of a particular brain area. The FDD scores have an intuitive interpretation with respect to disease state: as a patient’s disease progresses, their brain becomes less able to sustain complex fractal-like activity over time. Thus, their neural activity exhibits wider and less symmetric distributions of fractal scores.

Relative to simple whole-recording wideband fractal scores and oscillatory power, FDD scores are a much richer characterization of disease-relevant neural activity that capture changes in activity caused by neurodegeneration. As with simple whole-recording wideband fractal values and oscillatory power values, FDD scores carry information that helps distinguish healthy controls from Alzheimer’s Disease patients. Importantly, FDD scores demonstrate increased specificity and utility in identifying Alzheimer’s. As provided in the examples below, they have been used to discriminate AD from non-AD cases within mild cognitive impairment and dementia patient groups. Discriminating AD and non-AD cases in cognitively impaired or demented patients is a much more challenging task compared to discriminating healthy versus AD patients, and attempts of the former appear rarely in the academic literature. Success at this more difficult and clinically valuable task using FDD scores certifies the FDD technique as a qualitative advancement over related “ancestral” techniques.

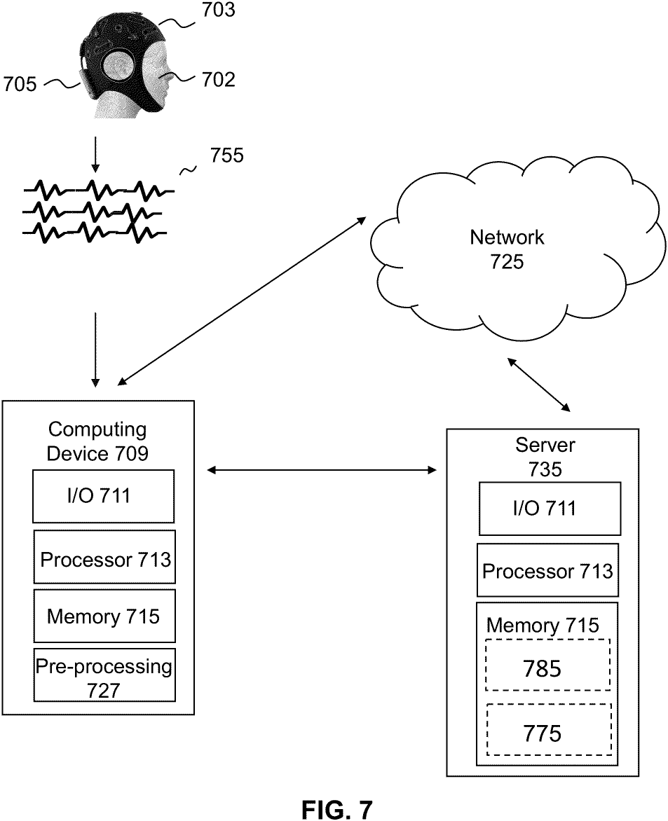

FIG. 7 shows select components of an exemplary system of the invention. The system an array 703 of EEG sensors that are positioned on the subject’s scalp, and a computing device 709.

Preferably, the array 403 of EEG sensors are incorporated into a cap such as the 21, 25, 32, and 64 channel waveguard™ EEG caps (ANT Neuro, Hengelo, Netherlands). Generally, EEG sensors are placed on bony structures on a subject’s scalp. By using an EEG cap, the sensors are correctly positioned on the subject’s scalp, eliminating the need to spend time carefully positioning the sensors.

The array/cap 703 may include an amplifier 705, which may include one or more filters. The amplifier 705 amplifies the raw potentials recorded from the sensors of the array/cap 703. The amplifier 705 may be connected to, or include, a programmable analogue/digital converter.

The array/cap 703 interacts (e.g., exchange data with) the computing device 709. The array/cap 703 may be connected to the computing device 709 via a wired connection. Alternatively, the array/cap 703 and computing device 709 exchange information using any combination of a local network (e.g., “Wi-Fi”), the internet, satellite, cellular data, or a short-range wireless technology, e.g., Bluetooth®. Neural data 755 from the subject is recorded using the array/cap 703 and transmitted to the computing device 709.

The computing device 409 may function as a remote or networked terminal that is in connection with a centralized computer system. Thus, the system may include one or more server computers 735 in communication with the computing device 709, preferably via connection with a network 725.

Each computing device 709 and server 725 includes a processor 713 coupled to a tangible, non-transitory memory 715 device and at least one input/output device 711. Thus, the system includes at least one processor 725 coupled to a memory subsystem 715. The components may be in communication over a network 725 that may be wired or wireless and wherein the components may be remotely located or located in close proximity to each other.

As shown in FIG. 7, the memory subsystem 715 of the server 709 includes one or more classifier 775 comprising one or more machine learning models. In certain aspects, the classifier 775 is hosted on a server 709 in communication with the computing device 709.

Processor refers to any device or system of devices that performs processing operations. A processor will generally include a chip, such as a single core or multi-core chip (e.g., 12 cores), to provide a central processing unit (CPU). In certain embodiments, a processor may be a graphics processing unit (GPU) such as a NVidia Tesla K80 graphics card from NVIDIA Corporation (Santa Clara, CA). A processor may be provided by a chip from Intel or AMD. A processor may be any suitable processor such as the microprocessor sold under the trademark XEON E5-2620 v3 by Intel (Santa Clara, CA) or the microprocessor sold under the trademark OPTERON 6200 by AMD (Sunnyvale, CA). Computer systems of the invention may include multiple processors including CPUs and or GPUs that may perform different steps of methods of the invention.

The memory subsystem 715 may include one or any combination of memory devices. A memory device is a device that stores data or instructions in a machine-readable format. Memory may include one or more sets of instructions (e.g., software) which, when executed by one or more of the processors of the disclosed computers can accomplish some or all of the methods or functions described herein. Preferably, each computer includes a non-transitory memory device such as a solid-state drive, flash drive, disk drive, hard drive, subscriber identity module (SIM) card, secure digital card (SD card), micro-SD card, or solid-state drive (SSD), optical and magnetic media, others, or a combination thereof.

The computing device 709 and server computer 735 may include an input/output device 711, which is a mechanism or system for transferring data into or out of a computer. Exemplary input/output devices include a video display unit (e.g., a liquid crystal display (LCD) or a cathode ray tube (CRT)), a printer, an alphanumeric input device (e.g., a keyboard), a cursor control device (e.g., a mouse), a disk drive unit, a speaker, a touchscreen, an accelerometer, a microphone, a cellular radio frequency antenna, and a network interface device, which can be, for example, a network interface card (NIC), Wi-Fi card, or cellular modem.

Using one or more I/O device or connection, the computing device 709 can transmit instructions to the EEG array 703. Recorded neural data can be stored in the memory subsystem of the computing device 709. In certain aspects, the neural data is transmitted to a remote computing device 709 from a server computer 735 via a network 725.

In certain aspects, the system further includes one or more means, in addition to the EEG array 703, to provide biometric data from a subject undergoing a cognitive assessment. For example, the system may include an imaging subsystem 731, such as a camera or specialized eye tracking device. The imaging subsystem 731 may be used, for example, to track a subject’s eye movement, or track micro-facial expressions (facial coding). Eye tracking can be used to provide additional data features analyzed by the familiarity classifier.

Surprisingly, the present Inventors have discovered that a specialized eye tracking device is not necessarily required for purposes of eye tracking. Rather, a digital camera, such as that integrated into a laptop serving as the computing device 709, can provide the necessary resolution for eye-tracking purposes.

Facial coding can be used, for example, to provide additional data regarding a subject’s emotional response during a cognitive assessment. This data can be used by the classifier to increase accuracy and specificity.

As shown in FIG. 7, one or more of the computing device 709 or server computer 735 includes a pre-processing module 727. The pre-processing module 727 performs one or more of the pre-processing steps as described herein. Pre-processing modules may include a combination of hardware (e.g., filters and amplifiers) and software components. In certain aspects, the computing device 709 includes a pre-processing module 727, which allows the computing device to transmit pre-processed neural data to a server computer 735 for analysis by a classifier hosted thereon. By performing at least part of the pre-processing on the computing device 709, less network bandwidth is required for transmitting the neural data 755 to the server computer 735. Neural data digitized and/or filtered from its raw, analogue state, is far less data intensive.

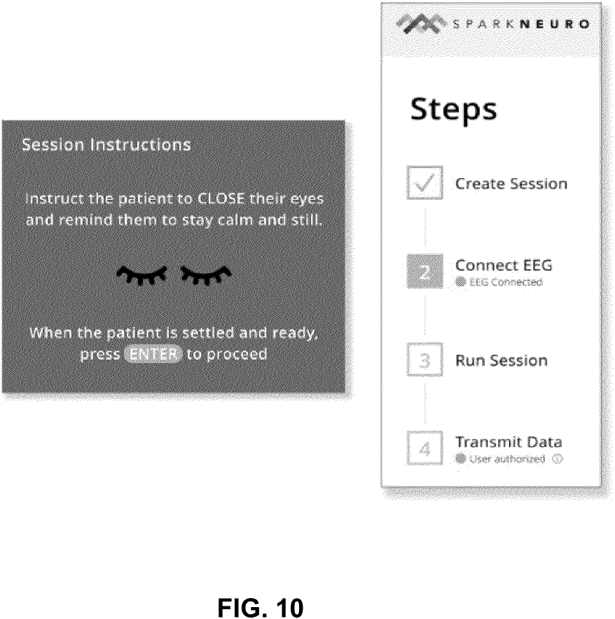

In certain aspects, the computing system 709 includes one or more applications for initializing and/or calibrating aspects of the classifier for a cognitive assessment. For example, as shown in FIG. 10, the computing system 709 may include instructions displayed to a subject undergoing an assessment regarding, for example, to close their eyes and remain still and how to affix the EEG cap 403. The instructions may task the subject with focusing on a certain spot on a screen, taking a physical action (e.g., standing or closing eyes), and the like. During this time, the computing system may, for example, calibrate the EEG array, an imaging subsystem (e.g., an eye tracking device) and/or obtain baseline neural data from the subject.

In certain aspects, neural data recorded during this time can be used to train a classifier. This training data can be used to train a classifier for use with other subjects. Alternatively or additionally, this training data is used to train the classifier that will analyze the subject’s neural data for a cognitive assessment.

In certain methods and systems, the classifier 755 includes one or more machine learning (ML) model. FDD scores obtained from EEG recordings using the systems and methods of the invention can be analyzed by these ML models of the classifier to provide accurate and insightful assessments of a subject’s AD status.

Systems and methods of the invention may produce an output that provides an assessment of a subject’s AD after a neurological assessment using the FDD-based techniques described herein.

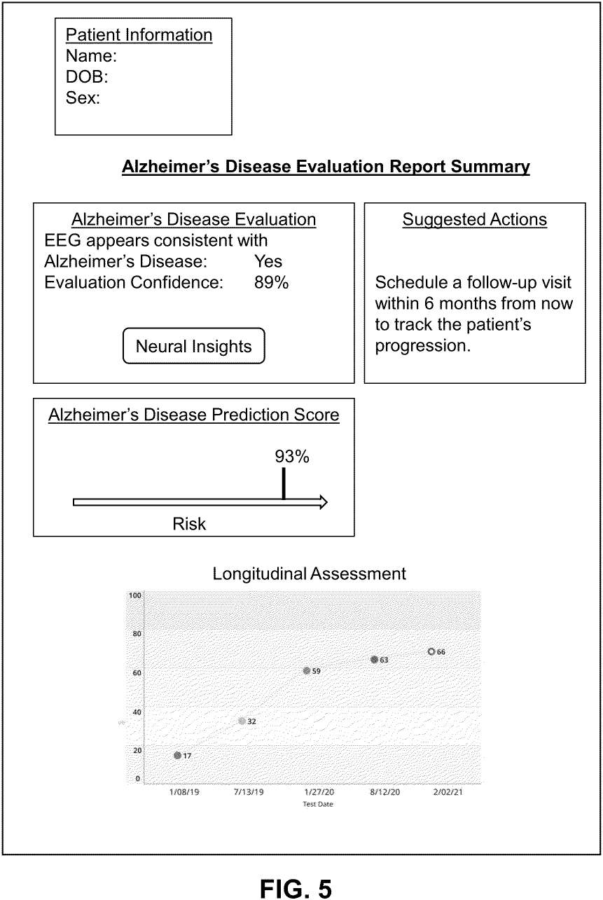

FIG. 5 provides an exemplary sample output produced by a system of the invention. This sample output represents a summary report that may be provided to a patient or clinician. As shown, a report may include biographical or medical history from a subject. In certain aspects, this information is obtained from an electronic medical record system.

The report includes an assessment of whether or not a subject exhibits signs of AD. In preferred aspects, this assessment is provided in a simple yes/no format. A confidence value may also be provided with the assessment. In certain aspects, the report further includes information about the metrics, measurements, and data underlying the assessment.

In certain aspects, the report includes a value on a continuous scale that may, for example, provide the probability that a subject has or will develop AD. In certain aspect, the report includes results from longitudinal assessments.

For example, in certain aspects, a user may receive the report electronically, and a link (shown as “Neural Insights” in FIG. 5) may be provided to guide a user to the metrics, measurements, and data underlying the assessment. For example, report may provide a section that exposes the features that were computed and fed into the algorithm. This section enables physicians to directly examine the patient data as well as providing some guidance around interpreting it with respect to disease state where that can be backed up by citations. This transparency empowers the physicians with greater insight into their patient’s brain function and provides them the opportunity to self-educate.

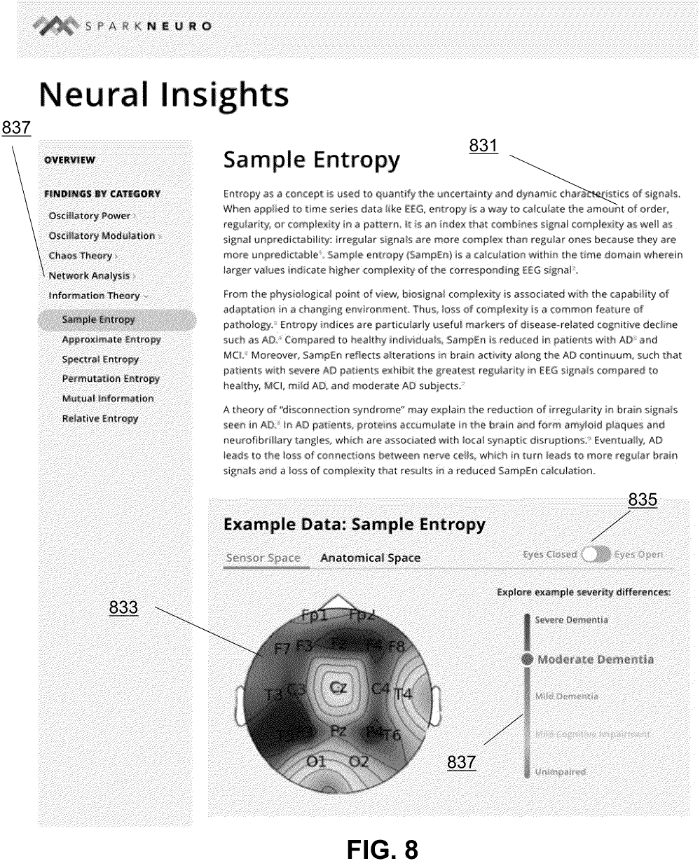

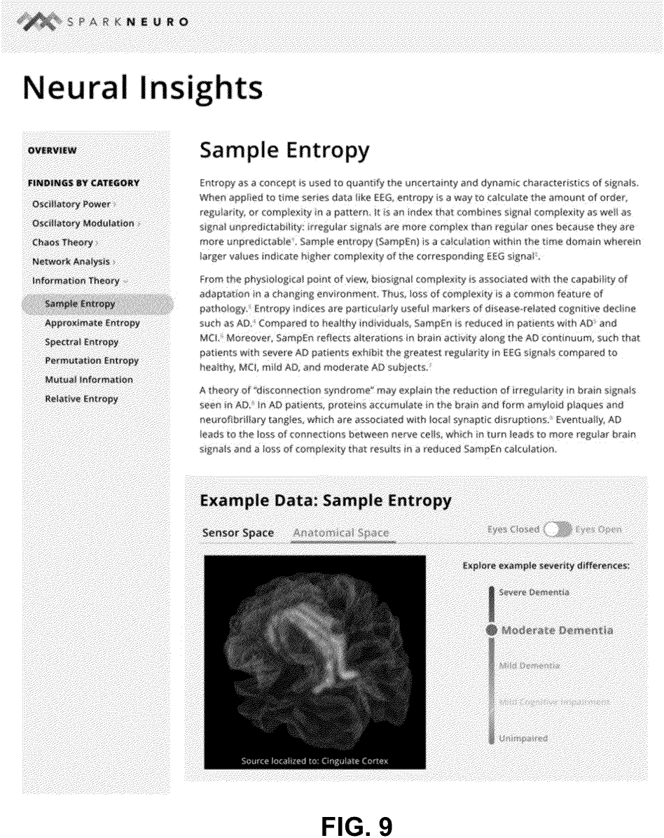

FIGS. 8-9 show exemplary sections of a report showing information regarding features. As shown, in certain aspects, the report provides a user with a list of features/measures 837 used by the classifier ranked by their importance for a subject, along with a quantification of how much influence that particular feature had on the assessment. Intuitively, features which generally have high predictive value in the model as well as features for which the patient had extreme values will be highlighted. In certain aspects, highlighting may indicate importance to top-level output for a particular. This may include highlighting only the largest contributors that cumulatively contributed 80% of the decision.

In this view, the hierarchy of viewing the features might first gloss major categories with a colored label to show how the classes of features might indicate disease/health. i.e., Network, Chaos, Information, Oscillation, and then little green/yellow/red lights for each. An alternative view lists features organized by category, rather than by ranking. Features are listed together in groups like Oscillatory, Chaos, Information Theory, Network, etc. It is possible that the number of features in the model that are exposed to the user for inspection become so extensive that the report will have to impose many layers of subcategorization on the features to organize them. This subcategorization may be applied to the category-based view of features. The view of features ranked by importance should include subcategories in the feature name. Feature-related keywords may have tooltips with a dictionary-style blurb explaining what they are, e.g., Delta power: “The strength of neural oscillations between 1 Hz - 4 Hz”; “Higuchi Fractal Dimension: “A chaos measure that captures the degree of scale-invariant structure in a signal across time”. In certain aspects, as shown in FIGS. 8-9, when a user selects a particular feature (metric) from the categorized of ranked lists, they are presented with the patient’s data, the importance of that feature category (e.g., sum of all SHAP values within that feature category), a description of the feature (metric) 831, and where possible, how it relates to the disease.

How the subject’s data is displayed for a particular feature may vary. As shown in FIG. 8, most features will be EEG-based and typically display the subject’s values and/or values indicating a particular AD diagnosis (837) or other condition (e.g., eyes open/closed 835), on a topographic head map 833 when a value is computed by sensor, or as shown in FIG. 9, in a 3D brain plot when a value is computed by anatomical location. Where z-scores are appropriate and available for a metric, users can toggle between metric scores and z-scores. For features that are summary statistics (such as FDD scores) across sensors or anatomical locations, the report still presents the individual values at each sensor/anatomical location, but indicates and emphasizes the summary statistic through the use of color and opacity.

There may be a challenge with performing repeated scans if a model used in the classifier learns or updates between scans. The underlying model will be improved over time, so the output (score, labels, etc.) for the exact same patient scan may also change with each update to the algorithm. This means that scores for a given subject scanned multiple times are only directly comparable when produced by the same version of the classifier. Using a patient’s AD status at different timepoints as an indicator of changes in their neurological health or cognitive status over time requires re-computing the AD status for each dataset from a subject (at Time 1, at Time 2, at Time 3 ...) using the current algorithm. Therefore, the report will provide freshly-computed AD status and any categorical labels (cognitive status, diseases, subtypes, etc.) at all timepoints using the current algorithm.

Where a report contains data from repeated scans and the categorical labels output for a previous scan change between what was reported when the scan was done and the current report due to an algorithm update, the report will note the label change and explain why it has changed.

Any of several suitable types of machine learning may be incorporated into one or more steps of the disclosed methods and systems. Classifiers of the invention may use machine learning approaches, which include neural networks, decision tree learning such as random forests, support vector machines (SVMs), association rule learning, inductive logic programming, regression analysis, clustering, Bayesian networks, reinforcement learning, metric learning, and genetic algorithms. One or more of the machine learning approaches (aka type or model) may be used to complete any or all of the method steps described herein.

For example, one model, such as a neural network, may be used to complete the training steps of autonomously identifying features in one or more subject’s neural data and associating those features with the AD status of a subject (e.g., during training or calibrating). Once those features are learned, they may be applied to test samples by the same or different models or classifiers (e.g., a random forest, SVM, regression) for the correlating steps during an AD assessment.

In certain aspects, features in FDD scores may be identified and associated with the AD status in a subject using one or more machine learning systems, and the associations may then be refined using a different machine learning system. Accordingly, some of the training steps may be unsupervised using unlabeled data while subsequent training steps (e.g., association refinement) may use supervised training techniques such as regression analysis using the features autonomously identified by the first machine learning system.

In decision tree learning, a model is built that predicts that value of a target variable based on several input variables. Decision trees can generally be divided into two types. In classification trees, target variables take a finite set of values, or classes, whereas in regression trees, the target variable can take continuous values, such as real numbers. Examples of decision tree learning include classification trees, regression trees, boosted trees, bootstrap aggregated trees, random forests, and rotation forests. In decision trees, decisions are made sequentially at a series of nodes, which correspond to input variables. Random forests include multiple decision trees to improve the accuracy of predictions. See Breiman, 2001, Random Forests, Machine Learning 45:5-32, incorporated herein by reference. In random forests, bootstrap aggregating or bagging is used to average predictions by multiple trees that are given different sets of training data. In addition, a random subset of features is selected at each split in the learning process, which reduces spurious correlations that can results from the presence of individual features that are strong predictors for the response variable. Random forests can also be used to determine dissimilarity measurements between unlabeled data by constructing a random forest predictor that distinguishes the observed data from synthetic data. Id.; Shi, T., Horvath, S. (2006), Unsupervised Learning with Random Forest Predictors, Journal of Computational and Graphical Statistics, 15(1): 118-138, incorporated herein by reference. Random forests can accordingly by used for unsupervised machine learning methods of the invention.

SVMs are useful for both classification and regression. When used for classification of new data into one of two categories, such as having a disease or not having the disease, a SVM creates a hyperplane in multidimensional space that separates data points into one category or the other. Although the original problem may be expressed in terms that require only finite dimensional space, linear separation of data between categories may not be possible in finite dimensional space. Consequently, multidimensional space is selected to allow construction of hyperplanes that afford clean separation of data points. See Press, W.H. et al., Section 16.5. Support Vector Machines. Numerical Recipes: The Art of Scientific Computing (3rd ed.). New York: Cambridge University (2007), incorporated herein by reference. SVMs can also be used in support vector clustering to perform unsupervised machine learning suitable for some of the methods discussed herein. See Ben-Hur, A., et al., (2001), Support Vector Clustering, Journal of Machine Learning Research, 2:125-137.

Regression analysis is a statistical process for estimating the relationships among variables such as features and outcomes. It includes techniques for modeling and analyzing relationships between multiple variables. Specifically, regression analysis focuses on changes in a dependent variable in response to changes in single independent variables. Regression analysis can be used to estimate the conditional expectation of the dependent variable given the independent variables. The variation of the dependent variable may be characterized around a regression function and described by a probability distribution. Parameters of the regression model may be estimated using, for example, least squares methods, Bayesian methods, percentage regression, least absolute deviations, nonparametric regression, or distance metric learning.

Association rule learning is a method for discovering interesting relations between variables in large databases. See Agrawal, 1993, Mining association rules between sets of items in large databases, Proc 1993 ACM SIGMOD Int Conf Man Data p. 207, incorporated by reference. Algorithms for performing association rule learning include Apriori, Eclat, FP-growth, and AprioriDP. FIN, PrePost, and PPV, which are described in detail in Agrawal, 1994, Fast algorithms for mining association rules in large databases, in Bocca et al., Eds., Proceedings of the 20th International Conference on Very Large Data Bases (VLDB), Santiago, Chile, September 1994, pages 487-499; Zaki, 2000, Scalable algorithms for association mining, IEEE Trans Knowl Data Eng 12(3):372-390; Han, 2000, Mining Frequent Patterns Without Candidate Generation, Proc 2000 ACM SIGMOD Int Conf Management of Data; Bhalodiya, 2013, An Efficient way to find frequent pattern with dynamic programming approach, NIRMA Univ Intl Conf Eng, 28-30 Nov. 2013; Deng, 2014, Fast mining frequent itemsets using Nodesets, Exp Sys Appl 41(10):4505-4512; Deng, 2012, A New Algorithm for Fast Mining Frequent Itemsets Using N-Lists, Science China Inf Sci 55(9): 2008-2030; and Deng, 2010, A New Fast Vertical Method for Mining Frequent Patterns, Int J Comp Intel Sys 3(6):333-344, the contents of each of which are incorporated by reference. Inductive logic programming relies on logic programming to develop a hypothesis based on positive examples, negative examples, and background knowledge. See Luc De Raedt. A Perspective on Inductive Logic Programming. The Workshop on Current and Future Trends in Logic Programming, Shakertown, to appear in Springer LNCS, 1999; Muggleton, 1993, Inductive logic programming: theory and methods, J Logic Prog 19-20:629-679, incorporated herein by reference.

Bayesian networks are probabilistic graphical models that represent a set of random variables and their conditional dependencies via directed acyclic graphs (DAGs). The DAGs have nodes that represent random variables that may be observable quantities, latent variables, unknown parameters or hypotheses. Edges represent conditional dependencies; nodes that are not connected represent variables that are conditionally independent of each other. Each node is associated with a probability function that takes, as input, a particular set of values for the node’s parent variables, and gives (as output) the probability (or probability distribution, if applicable) of the variable represented by the node. See Charniak, 1991, Bayesian Networks without Tears, AI Magazine, p. 50, incorporated by reference.

A neural network, which is modeled on the human brain, allows for processing of information and machine learning. A neural network includes nodes that mimic the function of individual neurons, and the nodes are organized into layers. The neural network includes an input layer, an output layer, and one or more hidden layers that define connections from the input layer to the output layer. The neural network may, for example, have multiple nodes in the output layer and may have any number of hidden layers. The total number of layers in a neural network depends on the number of hidden layers. For example, the neural network may include at least 5 layers, at least 10 layers, at least 15 layers, at least 20 layers, at least 25 layers, at least 30 layers, at least 40 layers, at least 50 layers, or at least 100 layers. The nodes of a neural network serve as points of connectivity between adjacent layers. Nodes in adjacent layers form connections with each other, but nodes within the same layer do not form connections with each other. A neural network may include an input layer, n hidden layers, and an output layer. Each layer may comprise a number of nodes.

The system may include any neural network that facilitates machine learning. The system may include a known neural network architecture, such as GoogLeNet (Szegedy, et al. Going deeper with convolutions, in CVPR 2015, 2015); AlexNet (Krizhevsky, et al. Imagenet classification with deep convolutional neural networks, in Pereira, et al. Eds., Advances in Neural Information Processing Systems 25, pages 1097-3105, Curran Associates, Inc., 2012); VGG16 (Simonyan & Zisserman, Very deep convolutional networks for large-scale image recognition, CoRR, abs/3409.1556, 2014); or FaceNet (Wang et al., Face Search at Scale: 90 Million Gallery, 2015), each of the aforementioned references are incorporated by reference.

Deep learning (also known as deep structured learning, hierarchical learning or deep machine learning) is a class of machine learning operations that use a cascade of many layers of nonlinear processing units for feature extraction and transformation. Each successive layer uses the output from the previous layer as input. The algorithms may be supervised or unsupervised and applications include pattern analysis (unsupervised) and classification (supervised). Certain embodiments are based on unsupervised learning of multiple levels of features or representations of the data. Higher level features are derived from lower-level features to form a hierarchical representation. Those features are preferably represented within nodes as feature vectors.

Deep learning by a neural network may include learning multiple levels of representations that correspond to different levels of abstraction; the levels form a hierarchy of concepts. In most preferred embodiments, the neural network includes at least 5 and preferably more than 10 hidden layers. The many layers between the input and the output allow the system to operate via multiple processing layers.

Deep learning is part of a broader family of machine learning methods based on learning representations of data. Neural data can be represented in many ways, e.g., as time series, by frequency, and/or in the spectral and/or time-frequency domains. The familiarity classifier can extract features from this data, which are represented at nodes in the network. Preferably, each feature is structured as a feature vector, a multi-dimensional vector of numerical features that represent some object. The feature provides a numerical representation of obj ects, since such representations facilitate processing and statistical analysis. Feature vectors are similar to the vectors of explanatory variables used in statistical procedures such as linear regression. Feature vectors are often combined with weights using a dot product in order to construct a linear predictor function that is used to determine a score for making a prediction.

The vector space associated with those vectors may be referred to as the feature space. In order to reduce the dimensionality of the feature space, dimensionality reduction may be employed. Higher-level features can be obtained from already available features and added to the feature vector, in a process referred to as feature construction. Feature construction is the application of a set of constructive operators to a set of existing features resulting in construction of new features.

The systems and methods of the disclosure may use convolutional neural networks (CNN) as part of the familiarity classifier. A CNN is a feedforward network comprising multiple layers to infer an output from an input. CNNs are used to aggregate local information to provide a global predication. CNNs use multiple convolutional sheets from which the network learns and extracts feature maps using filters between the input and output layers. The layers in a CNN connect at only specific locations with a previous layer. Not all neurons in a CNN connect. CNNs may comprise pooling layers that scale down or reduce the dimensionality of features. CNNs hierarchically deconstruct data into general, low-level cues, which are aggregated to form higher-order relationships to identify features of interest. CNNs predictive utility is in learning repetitive features that occur throughout a data set.

The systems and methods of the disclosure may use fully convolutional networks (FCN). In contrast to CNNs, FCNs can learn representations locally within a data set, and therefore, can detect features that may occur sparsely within a data set.

The systems and methods of the disclosure may use recurrent neural networks (RNN). RNNs have an advantage over CNNs and FCNs in that they can store and learn from inputs over multiple time periods and process the inputs sequentially.

The systems and methods of the disclosure may use generative adversarial networks (GAN), which find particular application in training neural networks. One network is fed training exemplars from which it produces synthetic data. The second network evaluates the agreement between the synthetic data and the original data. This allows GANs to improve the prediction model of the second network.

### EXAMPLES

### Example 1 — Success of FDD Technique Versus Other Measures

This Example demonstrates the utility of the FDD technique by constructing naive single-feature logistic models using FDD scores, relative oscillatory power, and wideband whole-recording fractal scores. The FDD features were selected from fully-developed classification models as high-importance features ranked by SHapley Additive exPlanations (SHAP) scores.

EEG data was collected from 240 patients as a real-world clinical sample from a memory clinic within a major health system. Pathology and cognitive status diagnoses were made via consensus from a panel made up of a clinical dementia neurologist, a geriatric psychiatrist, and a neuropsychologist. Mixed-disease was common across the sample. A minority of patients had their Alzheimer’s Disease status biomarker confirmed via tau or amyloid PET.

The tables below report the single-feature logistic model AUC-ROC scores for different classification tasks. For a given FDD, related oscillatory power and fractal comparison models were constructed with features computed from the same EEG timeseries data the FDD was computed from. Thus, for example, if a given FDD score is the standard deviation of the distribution of Higuchi fractal dimension scores computed within theta band activity at EEG sensor O2, then the comparison models would use relative theta power values and Higuchi fractal dimension scores computed from the same EEG data at sensor O2.

An AUC-ROC curve of a logistic model for each measure (FDD, power, and fractal) was produced.

TABLE 1

— AUC-ROC curve results for discriminating AD status in patients with subjective cognitive impairment versus those with having dementia with AD

| Measure | FDD technique | Whole-recording | Relative Oscillatory Power |
|---|---|---|---|
| Measure 1 | 0.715 | 0.533 | 0.505 |
| Measure 2 | 0.719 | 0.659 | 0.650 |

TABLE 2

AUC-ROC curve results for discriminating AD status in patients with subjective cognitive impairment versus those with mild cognitive impairment with AD

| Measure | FDD technique | Whole-recording | Relative Oscillatory Power |
|---|---|---|---|
| Measure 1 | 0.772 | 0.624 | 0.401 |
| Measure 2 | 0.743 | 0.581 | 0.423 |
| Measure 3 | 0.740 | 0.621 | 0.620 |

TABLE 3

AUC-ROC curve results for discriminating AD status in patients with mild cognitive impairment due to probable AD versus those with mild cognitive impairment due to non-AD causes

| Measure | FDD technique | Whole-recording | Relative Oscillatory Power |
|---|---|---|---|
| Measure 1 | 0.760 | 0.514 | 0.496 |
| Measure 2 | 0.756 | 0.570 | 0.583 |
| Measure 3 | 0.746 | 0.562 | 0.475 |

TABLE 4

AUC-ROC curve results for discriminating AD status in patients with dementia caused by AD versus those with dementia due to other causes

| Measure | FDD technique | Whole-recording | Relative Oscillatory Power |
|---|---|---|---|
| Measure 1 | 0.831 | 0.460 | 0.522 |
| Measure 2 | 0.824 | 0.521 | 0.540 |
| Measure 3 | 0.854 | 0.682 | 0.617 |

As provided in the tables showing the AUC-ROC curves, the FDD technique shows consistent superiority over existing techniques when classifying patients with subjective cognitive impairment versus those with Alzheimer’s Disease at the mild cognitive impairment or dementia stages. FDD demonstrates remarkable utility and surprising superiority in discriminating Alzheimer’s patients from non-Alzheimer’s patients when comparing within a level of cognitive impairment. This is in sharp contrast to related fractal and oscillatory metrics, which in these cases carry next to no information about disease status. The AUC-ROC curves show that the FDD technique is superior and carries unique information about Alzheimer’s Disease relative to related known measures.

### Example 2 — Healthy Versus Mild AD Subjects

This Example demonstrates the utility of the FDD technique for detecting the presence of Alzheimer’s disease by developing an XGBoost classifier capable of distinguishing between healthy controls and mild AD subjects using FDD scores and other EEG features. Cross-validation was used for both model development and for characterizing model performance. ML models and features (e.g., FDD scores) used by the models for assessing AD status are selected using only the “train” portion of train/test splits. Test data was not used to inform decisions about selecting specific ML model architectures, hyperparameters, or selected features. This helped to prevent overfitting and yielded robust estimates of generalization performance. Summaries were computed from test data.

This Example used a different patient sample from Example 1. The patient data in this sample was an archival dataset collected at a university clinic. It included 24 healthy controls and 24 mild AD patients, all of which were included in the analysis.

Along with ROCAUC, an optimal sensitivity, specificity, and accuracy for the point on the ROC curve that maximizes Youden’s j was determined. When used to assess this healthy versus mild AD patient example, the classifier’s performance provides a ROCAUC = 98%, sensitivity = 90%, specificity = 99%, and accuracy = 96%. The classifier performance was significantly better than chance p < 0.001.

A semi-automated feature selection method was used to train the model. Along with FDD features, the selection process used a variety of feature categories to choose from that appear in academic literature. These feature categories included wideband whole-recording fractal dimension scores, relative power, absolute power, amplitude modulation, entropy measures, functional connectivity network metrics etc. The automated feature-selection process selected many FDD features as indicators of AD status. SHAP scores were used to rank features by how important and useful they were for detecting AD. The scores reveal that a predominance of the top 10 features were FDD features. This supports the utility of the FDD technique for assessing the AD status of a subject.

### Example 3 — Subjective Cognitive Impairment Versus Mild AD Subjects

This Example demonstrates the utility of the FDD technique for detecting the presence of Alzheimer’s disease by developing a XGBoost classifier capable of distinguishing between patients with subjective cognitive impairment and mild AD patients using FDD and other EEG features. The classifier-development approach is as described in Example 2. The dataset is described in Example 1 (240 total patients, data collected at memory clinic, panel consensus diagnosis).

In this subjective cognitive impairment vs mild AD patient example, the classifier’s performance was ROCAUC = 89%, sensitivity = 76%, specificity = 87%, accuracy = 84%. The classifier performance was significantly better than chance p < 0.001.

As was the case in Example 2, the automated feature-selection process selected many FDD features as indicators of AD status. When SHAP scores were used to rank features by how important and useful they were for detecting AD using, again, the predominance of the top 10 were FDD features.

### Example 4 — AD Vs Non-AD Cases in Mild Cognitive Impairment (MCI) and Dementia Subjects

This Example demonstrates the utility of the FDD technique for detecting the presence of Alzheimer’s disease by developing a XGBoost classifier capable of distinguishing between impaired patients (MCI and dementia) with an Alzheimer’s diagnosis, versus impaired patients (MCI and dementia) due to any and all causes other than Alzheimer’s disease. This is a particularly difficult challenge, as all patients (including those with an AD diagnosis) had multiple diagnoses that can contribute to impairment (such as dementia with Lewy bodies, diabetes, depression, Parkinson’s disease dementia, and vascular dementia etc.). The classifier-development approach is as described in Example 2. The dataset is the same described in Example 1 (240 total patients, data collected at memory clinic, panel consensus diagnosis).

In this example where the task was to detect which impaired patients have AD and which do not, the classifier’s performance was ROCAUC = 82%, sensitivity = 72%, specificity = 87%, accuracy = 80%. The classifier performance was significantly better than chance p < 0.001.

As was the case in Example 2, the automated feature-selection process selected many FDD features as indicators of AD status. SHAP scores were again used to rank features by how important and useful they were for detecting AD. The predominance of the top 10 most useful and informative features for detecting AD were again FDD features, as they were in Examples 2 and 3.

### Example 5 - A Novel Machine Learning Technique Using Resting-State EEG to Assess And Monitor Cognitive Impairment

### Background

Monitoring changes in cognitive impairment is an important aspect of care for aging patients. Traditionally, this is done with pen-and-paper assessments such as the Mini-Mental State Exam (MMSE). EEG is a low-cost and widely available neurophysiology tool that provides a data-rich measure of brain health, but signal complexity has limited its impact. Advances in machine learning bring new possibilities to decode those complex signals and leverage EEG in patient care. The present Example shows the development and use of novel EEG features for assessing brain health with the Fractal Dimension Distributions (FDD) technique. FDD and other EEG metrics were used to assess patients’ cognitive function via a continuous score (a Cognitive Impairment Index, or CII). The diagnostic value of the EEG features were validated by comparing an FDD-based model against reduced models lacking the EEG features.

### Methods

The patient sample (N=97, mean (sd) age = 69.1 (11.0), 52.6% female) was collected at the Pacific Brain Health Center. Diagnostic categories included subjective cognitive impairment (n=44), mild cognitive impairment (n=26), and dementia (n=27). Mixed disease (AD, DLB, FTD, vascular, diabetes) was common in the impaired sample, and 39.6% were diagnosed with AD. A continuous (CII) model trained on patients’ MMSE scores (range = 11 to 30, mean = 26.6) using a gradient boosting model with Fractal Dimension Distributions and other EEG features computed from clinical wake EEGs was developed. Model development and testing took place within a rigorous nested cross-validation (NCV) procedure to prevent overfitting. Performance was compared to reduced (no-EEG features) models using the non-parametric Wilcoxon sign-rank test.

### Results

The approach showed a Mean Absolute Error of 1.98 (sd = 0.28) across 5 test folds in the NCV outer loops, significantly outperforming both the baseline intercept-only model (p = 0.031) and the demographics (age and years of education) model (p = 0.031).

### Conclusion

These preliminary results suggest that the CII derived from machine-learning and quantitative EEG using FDD is a viable approach to assess and track patients’ cognitive impairment regardless of disease status across all impairment levels, raising the possibility that a brief EEG scan could shed light on patient health normally accessible only through extensive cognitive and neuropsychological testing.

### Example 6 — Fractal Dimension Distributions (FDD) Of Resting-State Eeg Improve Detection Of Dementia And Alzheimer’s Disease

### Background

Across a large number of resting-state electroencephalography (EEG) studies, dementia is associated with changes to the power spectrum and fractal dimension. This Example describe a novel method used to examine changes in fractal dimension over time and within frequency bands. This method incorporates Fractal Dimension Distributions (FDD), which combine spectral and complexity information. In this study, resting-state EEG data was recorded from patients with subjective cognitive impairment (SCI) or dementia and FDD metrics from the data were compared with standard Higuchi and Katz fractal dimension metrics. FDD revealed larger group differences detectable at greater numbers of EEG recording sites. Moreover, linear models using FDD features had lower AIC and higher R² than models using standard full time-course measures of fractal dimension. FDD metrics also outperformed the full time-course metrics when comparing SCI with a subset of dementia patients diagnosed with Alzheimer’s disease (AD). As shown, FDD offers unique information beyond traditional full time-course fractal analyses, and can be used to identify dementia and to distinguish AD dementia from non-AD dementia.

Most AD patients first experience a stage of mild cognitive impairment (MCI), where memory loss and other cognitive changes are pronounced enough to register on clinical assessments, but not so extreme as to interfere with independent daily functioning. Before being diagnosed with MCI, some patients report worsening cognitive abilities despite scoring within healthy ranges on clinical assessments (Reisberg et al., 2008); this condition is referred to as subjective cognitive impairment (SCI). AD is primarily diagnosed through clinical cognitive assessments (e.g., McKhann et al., 2011), which may not always catch early-stage cognitive impairment or distinguish between different forms of dementia (Arevalo-Rodriguez et al., 2015).

AD patients present with a number of EEG abnormalities, one of which is reduced signal complexity (Al-Nuaimi et al., 2018; Sun et al., 2020; Yang et al., 2013). Complexity in brain signals arise from interacting neural circuits operating over multiple spatial and temporal scales. Fractal dimension (FD) is a nonlinear measure that expresses how the details of a self-similar form are altered by the scale at which they are measured (Mandelbrot, 1967). In this Example, both the Katz Fractal Dimension (KFD) and the Higuchi Fractal Dimension (HFD) methods were used. KFD calculates the fractal dimension by comparing distances along the waveform (Katz, 1988), whereas HFD approximates the box-counting dimension in time-series data by repeatedly downsampling a waveform and comparing the length of the subsampled waveforms to the downsampling factor (Higuchi, 1988). Though KFD tends to underestimate FD, KFD is sometimes better at discriminating between different brain states than HFD (Lau et al., 2022)

Diminished HFD and KFD are observed in the EEG and magnetoencephalography (MEG) of individuals with AD and dementia (Al-Nuaimi et al., 2017; Gómez et al., 2009; Gómez & Hornero, 2010; Nobukawa et al., 2019; Smits et al., 2016). While EEG signal complexity drops after age 60 even in the absence of disease, AD is associated with a decrease in FD beyond what is observed in healthy, age-matched controls (Gómez et al., 2009). HFD and KFD have been used in conjunction with machine learning algorithms to distinguish between AD and healthy controls with high accuracy and specificity. These algorithms, are improved by computing FD within distinct frequency bands (e.g. delta, theta, alpha) rather than in broadband EEG.

As shown in the present Example, the predictive capability of EEG is further improved by analyzing the distribution FD over the course of an EEG session. Some HFD and KFD algorithms utilize the entire time series, meaning any changes in FD over time are lost. Some previous work calculated FD within moving windows but discarded information about potential changes in FD over time by averaging the resulting values together. However, the distribution of FD over time contains valuable information. FDD has the power to distinguish dementia from SCI patients by analyzing the distribution of FD over time. Rather than assessing FD using the full EEG time-course, FDD slides a moving window across the full time-course, computes FD (HFD or KFD) within each window, and then summarizes the distribution of FD values across time-windows (e.g., mean, standard deviation, etc.).

### Materials and Methods

### Study Population

All patients were adults over the age of 55 who visited a specialty memory clinic (Pacific Brain Health Center in Santa Monica, CA) for memory complaints. Adults were evaluated by a dementia specialist during their visit. Evaluations included behavioral testing and EEG recordings. Patients with SCI or dementia were selected retrospectively by reviewing charts for patients seen between July 2018 and February 2021.

Full data was available from 148 adults (91 female, Age M=71.3 years, SD=7.5). Groups were divided into adults diagnosed with SCI (N=97, 59 [60.8%] female, Age M=70.2, SD=7.1) or dementia (N=51, 32 [62.7%] female, Age M=73.7, SD=7.8).

Within the dementia group, 38 individuals were diagnosed with AD (26 [68.4%] female, Age M=74.2, SD=7.1). The remaining individuals were diagnosed with Lewy body dementia (n=4), vascular dementia (n=2), frontotemporal dementia (n=2), Parkinson’s disease (n=2), or unknown (n=3) All procedures aligned with the Helsinki Declaration of 1975 and were approved by the Institutional Review Board at the St. John’s Cancer Institute.

### Clinical Diagnosis

Patient diagnosis was based on consensus of a panel of board-certified dementia specialists. Diagnoses utilized standard clinical methods for neurological examinations, cognitive testing (MMSE (Folstein et al., 1975) or MoCA (Nasreddine et al., 2005)), clinical history (e.g. depression, diabetes, head injury, hypertension), and laboratory testing (e.g., thyroid stimulating hormone levels, rapid plasma regain (RPR) testing, vitamin B-12 levels).

Cognitive impairment was diagnosed on the basis of the MMSE (or MoCA scores converted to MMSE (Bergeron et al., 2017)). MCI was diagnosed according to the criteria established in (Langa & Levine, 2014), and distinguished from dementia on the basis of preserved functional abilities and independence together with a lack of significant impairment in occupational or social functioning. SCI was diagnosed based on subjective complaints without evidence of MCI.

### EEG Collection

EEG data were recorded using a 19-channel eVox System (Evoke Neuroscience) at the Pacific Neuroscience Institute. Electrodes were positioned in a cap according to the international 10-20 system (Fp1, Fp2, F7, F3, Fz, F4, F8, T7, C3, Cz, C4, T8, P7, P3, Pz, P4, P8, O1, and O2). Data were collected at 250 Hz while patients completed two resting-state recordings — five minutes each of eyes closed and eyes open — and a 15 minute Go/No-Go task. This study used only the eyes-closed resting-state data.

### EEG Preprocessing

EEG data were re-referenced offline to the average of all channels. Jump artifacts were identified as global field power more than 10 standard deviations from the mean, over a maximum of 10 sequential samples. These artifacts were removed and replaced with a linear interpolation between the nearest samples that were not contaminated by jump artifacts, computed separately for each channel. Ocular artifacts were removed with the aid of Independent Component Analysis (ICA). 18 ICA components were extracted, then compared to a template of stereotypical ocular artifacts. After the ocular artifact was removed, ICs were projected back into sensor space.

For the broadband analysis, data was filtered with a 1-50 Hz zero-phase finite impulse response bandpass to help attenuate line noise. In the banded analysis, separate bandpass filters were applied for the delta (1-4 Hz), theta (4-8 Hz), alpha (8-13 Hz), beta (13-30 Hz), and gamma (30-50 Hz) frequency bands. After filtering, data was segmented into 1 s duration epochs, with a 0.5 s overlap.

### Fractal Dimension

KFD and HFD were calculated using the AntroPy package (version 0.1.4). Higuchi’s method starts by subsampling a time-series across progressive smaller time scales. Thus, HFD depends on both the number of sample points (N) and the parameter kₘₐₓ, which sets the upper limit on the number of time intervals (Higuchi, 1988). Some early work suggested using kₘₐₓ= 6 for time series with 40-1000 points (Accardo et al., 1997). Other approaches suggest calculating HFD across multiple kₘₐₓ values and identifying the value of kₘₐₓ at which HFD plateaus (Doyle et al., 2004; Wajnsztejn et al., 2016). However, HFD is not guaranteed to plateau. In the present example an estimated optimal kₘₐₓ based on the length of the time-series of the full time-course or 1 s windows was used, as described in Wanliss & Wanliss, (2022), which is incorporated herein by reference. The indicated kₘₐₓ = 108 for the full sample and kₘₐₓ = 25 for 1 s windows.

### Full Time-Course Fractal Dimension

The full time-course fractal dimension was calculated by applying Katz’s method or Higuchi’s method (with kₘₐₓ= 108) to the entire resting-state EEG recording. This analysis reflects the way prior studies computed FD (Al-Nuaimi et al., 2017; Amezquita-Sanchez et al., 2019).

### Fractal Dimension Distributions (FDD)

For the fractal dimension distributions, first the data was segmented using moving windows 1 s moving windows, with a 0.5 s overlap (see below for discussion of other window sizes). Within each window, the KFD or HFD (kₘₐₓ = 25) was extracted within each window. Finally, the distribution of KFD or HFD values were summarized across windows using the mean and standard deviation.

### Group Differences

The fractal dimension measures were compared between the SCI and dementia. A threshold-free cluster enhancement (TFCE) was used to estimate the difference at each channel (Mensen & Khatami, 2013; Smith & Nichols, 2009). TFCE is a non-parametric technique that computes channel-level statistics which take into account the strength of the difference at each channel and the spatial extent of any clusters that exist in the data. TFCE-adjusted t-statistics were calculated based on 10,000 simulations, then group differences were visualized by projecting the t-statistics to the scalp with a bilinear interpolation in MNE (version 1.0.0), and individually significant channels were identified using alpha corresponding to corrected p < 0.05. In order to understand whether non-AD dementia and AD might be associated with different patterns of FD, this analysis was repeated comparing SCI to AD.

### Logistic Regressions

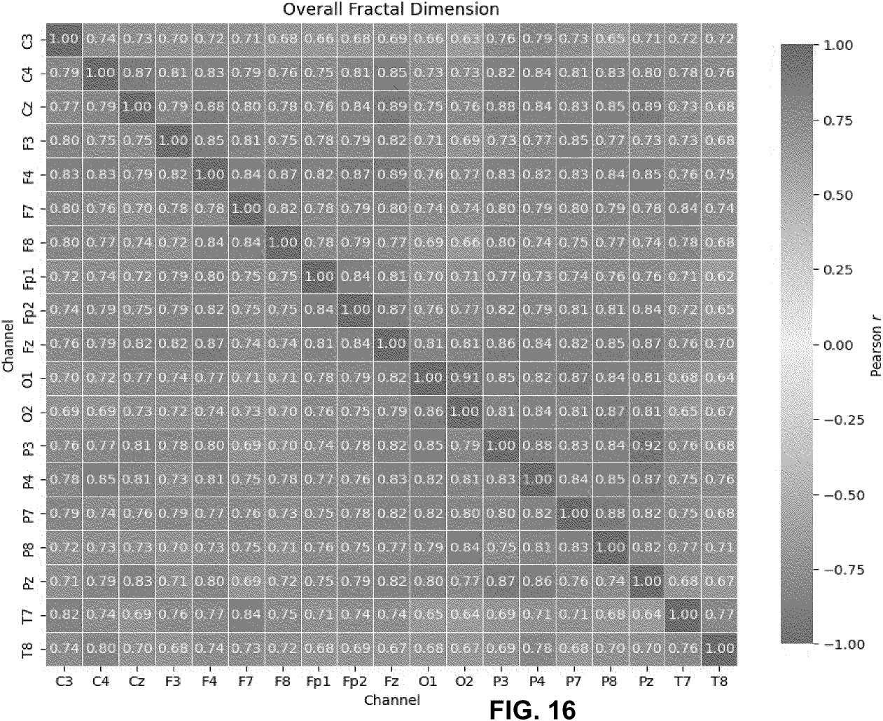

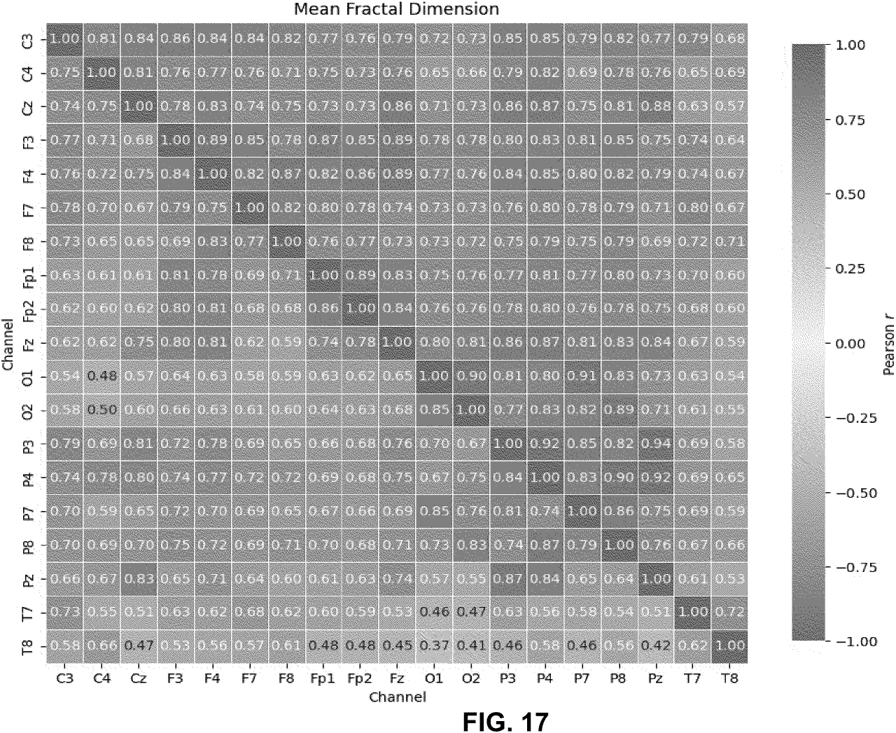

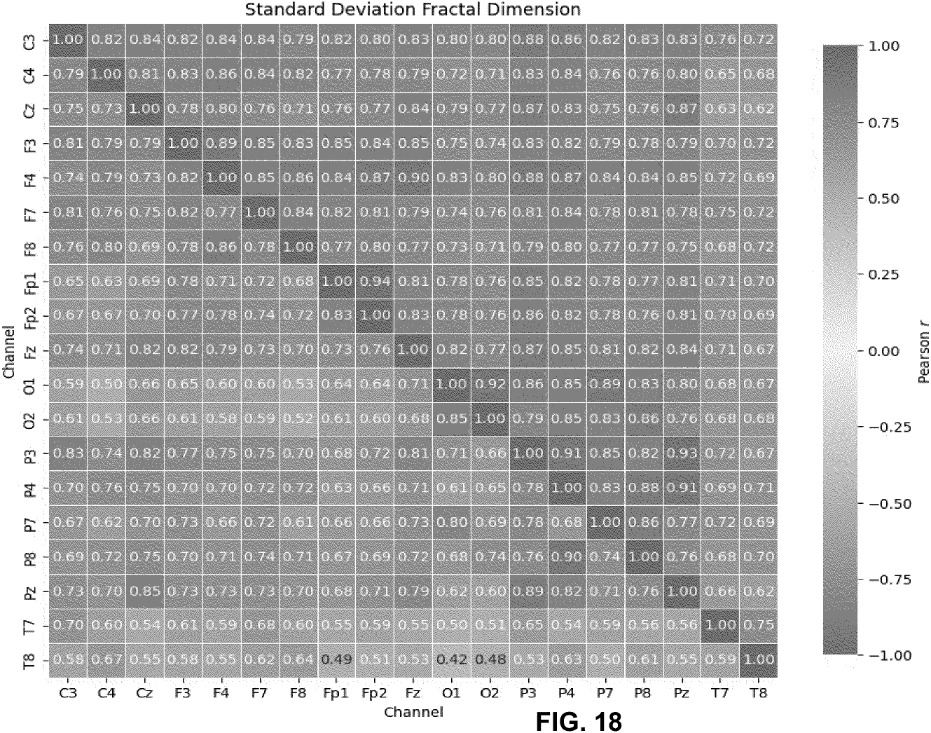

The TFCE analyses test whether FD metrics in individual channels carry information about dementia. As a complementary analysis, logistic regressions were used to test how information can be combined across channels to predict dementia (or AD). Cognitive status was regressed on FD metrics in all channels, using either full time-course FD or FDD features. All models included age as a covariate. Fractal scores across channels were correlated (see FIGS. 16-18), so regularized Least Absolute Shrinkage and Selection Operator (LASSO) regressions were used. For each set of predictors, a series of logistic regressions were fit, with L1 penalty values (alpha) ranging from 0 to 10 in 0.05 step increments. The model with the lowest Akaike Information Criterion (AIC) was selected for that set of predictors.

Model fit was assessed using a chi-square likelihood ratio test (LRT) comparing models with fractal features to a model with age as the only predictor. The LRT is inappropriate to directly compare models using full time-course FD to models using FDD features since they are non-nested. Instead, AIC was used, with lower AIC values indicating a better model. For each model Tjur’s coefficient of determination was also calculated to obtain pseudo-R², which reflects how well the model separates the two classes of patients, with 0 reflecting no separation and 1 reflecting complete separation (Tjur, 2009).

### Results

The model with age as the only predictor was a better fit to the data than an intercept-only null model (X²(1)=7.59, p=0.006).

### I. FDD Differentiates Dementia Better Than Full Time-Course Fractal Dimension

Previous studies were replicated by looking for differences in full time-course HFD and KFD between SCI and dementia (e.g., Al-Nuaimi et al., 2017). In the broadband data, group differences in full time-course HFD and full time-course KFD were in the expected direction, but did not reach statistical significance. Three electrodes did show a trend towards lower HFD in the Dementia group (O1 t = -1.37, p = 0.099; Pz t = -1.40, p = 0.099, and P3 t = -1.44, p = 0.089).

The FDD features were then examined by calculating the mean and standard deviation of HFD and KFD across windows. In the broadband data, the mean of windowed HFD did not significantly differ between groups. In contrast, the standard deviation of windowed HFD was significantly higher in the Dementia group at every electrode (smallest difference at T8, t=1.92, p=0.020, largest difference at T7, t=2.89, p<0.001). Further, the standard deviation of windowed KFD was significantly higher in the Dementia group at all but five electrodes (O1, O2, P3, P7, Cz; all p > 0.08).

A logistic regression model using full time-course HFD showed better performance than a model using only patients’ age, and both of these were outperformed by a model using FDD features (Table 5; ΔAIC = -16.7, ΔR² = 0.16). Similarly, a model using FDD features based on KFD outperformed a model using the full time-course KFD (Table 6; ΔAIC = -12.7, ΔR² = 0.24). Interestingly, the lowest AIC across models using full time-course KFD was a model in which LASSO regularization set all channel coefficients to 0, retaining only age; this indicates that KFD obtain from the full time-course did not contain enough unique information relating to dementia to be included in the model.

### II. FDD is More Informative Than Full Time-Course Fractal Dimension In Most Frequency Bands

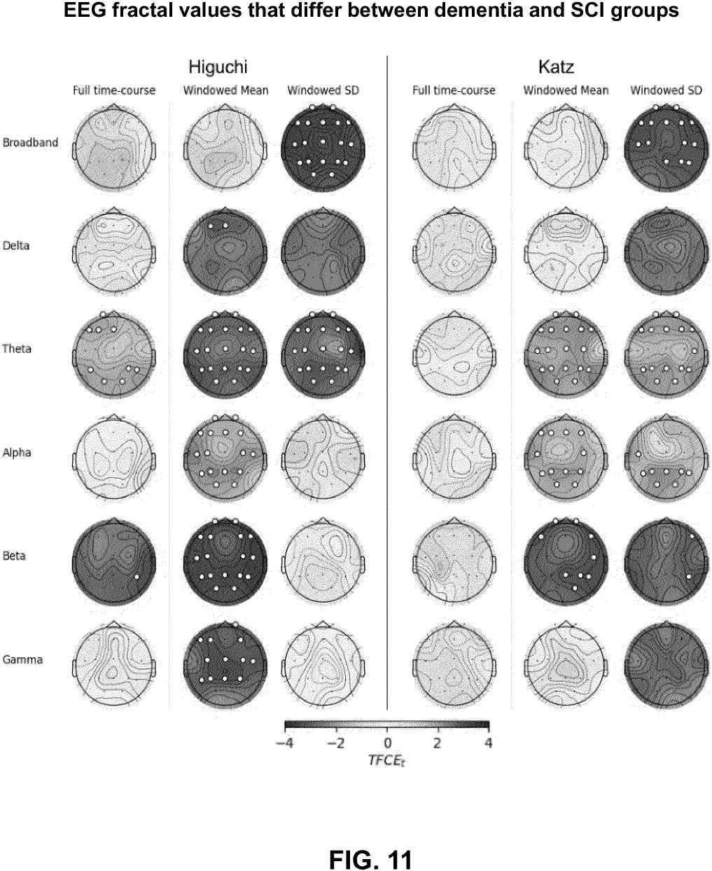

The fractal dimensions between SCI and dementia were compared within five frequency bands, calculating either the full time-course FD or FDD. FIG. 11 provides scalp plots showing differences (Dementia - SCI) calculated using threshold-free cluster enhancement (TFCE) for Higuchi fractal dimension (left) or Katz fractal dimension (right). Electrodes with significant differences (TFCE FWEp < 0.05) are marked with white circles.

As shown, in every band, more scalp locations showed significant group differences using Higuchi FDD calculated from full time-course HFD, particularly the windowed mean (FIG. 11, left). There were no significant differences in any band using the full time-course KFD (all ps > 0.187). Using the Katz FDD, however, several clusters of electrodes showed significant group differences in the theta, alpha, and beta bands.

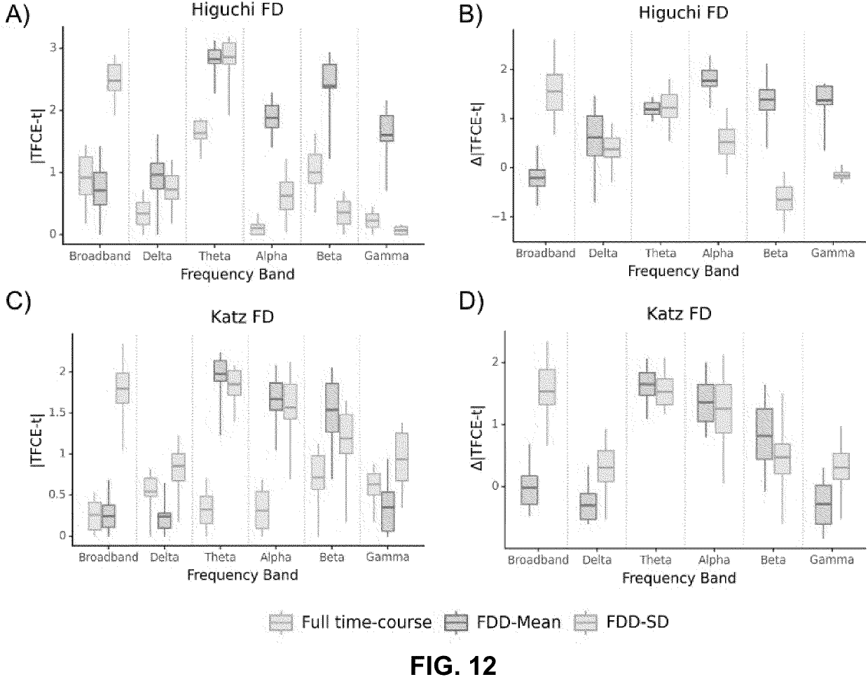

In order to visualize the extent to which FDD and full time-course FD discriminate between SCI and dementia, the absolute TFCE-t statistic averaged across channels was plotted for the full time-course FD and FDD mean and standard deviation. FIG. 12 shows these values for HFD and KFD for each frequency band. In FIG. 12, absolute TFCE-t statistic averaged across channels for group differences (Dem - SCI) in each frequency band using Higuchi (A) or Katz FD (C). Difference between FDD metrics and full time-course FD in each frequency band (B and D). Box outlines show the interquartile range, central line shows the median, and whiskers extend to minimum and maximum value.

Then the full time-course FD values were subtracted from the corresponding FDD metrics to obtain a numerical measure of the increase or decrease in group difference at each channel - positive values indicate a larger difference using FDD features compared to full time-course FD features. These relative TFCE values were plotted in each frequency band for HFD (FIG. 12) and KFD (FIG. 12).

As in the broadband analysis, logistic LASSO regressions were then used to test whether using FDD features leads to more accurate models than full time-course FD. For both Katz and Higuchi methods, models using FDD outperformed models using full time-course FD, though the effect was stronger for HFD (Table 5; average ΔAIC = -7.9, ΔR² = 0.15) than KFD (Table 6; average ΔAIC = -3.5, ΔR² = 0.08). The logistic regression using FDD with Higuchi in the delta band had lower AIC but did not have higher R² than the model using full time-course HFD (ΔAIC = -2.7, ΔR² = -0.03). In contrast, regressions using Higuchi FDD features were universally better in the theta (ΔAIC = -15.3, ΔR² = 0.32), alpha (ΔAIC = -15.4, ΔR² = 0.38), and beta (ΔAIC = -6.5, ΔR² = 0.14) bands. The model with full time-course HFD had a slightly lower AIC than the model with FDD in the gamma band (ΔAIC = 0.3, ΔR² = -0.08).

With Katz’s method, models using FDD had lower AIC and larger R² than models using full time-course KFD in delta (ΔAIC = -7.8, ΔR² = 0.17) and beta bands (ΔAIC = -9.2, ΔR² = 0.14). The model using FDD features had lower AIC and slightly larger R² in the alpha band (ΔAIC = -0.9, ΔR² = 0.01). In the theta band, the model using FDD features produced a smaller AIC, but worse R² (ΔAIC = -4.7, ΔR² = -0.04). As with HFD, in the gamma band, the model using full time-course KFD outperformed the model using FDD (ΔAIC = 5.0, ΔR² = 0.10).

Across all models comparing Dementia and SCI, the two with the lowest AIC and highest R² used Higuchi FDD features in the alpha band (AIC=166.6, R² = 0.644, X² (25)=66.42, p < 0.001) or theta band (AIC=159.6, R² = 0.592, X²(23)=69.44, p < 0.001).

### III. FDD Differentiates Alzheimer’s Disease Better Than Full Time-Course Fractal Dimension

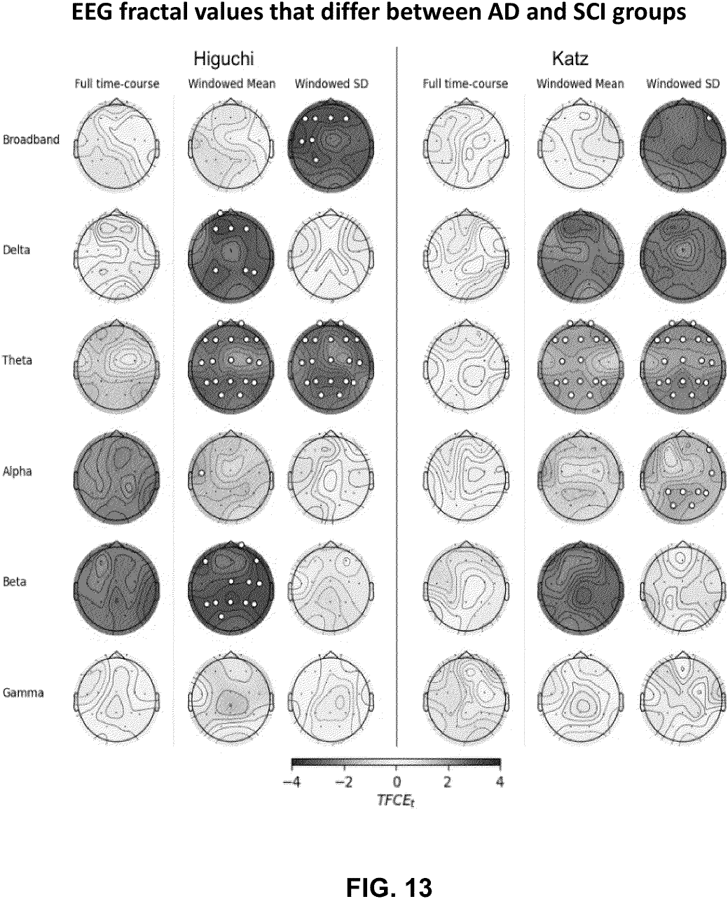

The previous analyses demonstrate that the distribution FD carries additional information about dementia beyond the information carried by standard full time-course FD. Next, it was assessed whether this distributional information also helps to specifically distinguish between SCI and dementia due to Alzheimer’s disease (AD). There were no significant differences between SCI and AD in full time-course HFD or KFD when estimated from broadband data (FIG. 13; all p > 0.3). There were also no significant differences in mean HFD or mean KFD at any electrode (all p > 0.2). However, KFD SD was significantly higher in the AD group at F8 (t=1.71, p=0.037) and HFD SD was higher at left and frontal sites, including F4, Fz, F3, F7, T7, C3 and P3.

FIG. 13 provides the resulting scalp plots that show differences (AD - SCI) calculated using threshold-free cluster enhancement (TFCE) for Higuchi fractal dimension (left) or Katz fractal dimension (right). Electrodes with significant differences (TFCE FWEp < 0.05) are marked with white circles.

The logistic regression using age to predict AD status was a significantly better fit than the intercept-only model (X²(1)=7.22, p=0.007). Again, while full time-course HFD improved model fit relative to an age-only model, models with FDD features fit the data even better (Table 4; ΔAIC = -5.4, ΔR² = 0.13). Furthermore, the model using Katz FDD features outperformed the model using full time-course KFD (ΔAIC = -10.7, ΔR² = 0.42).

### IV. FDD Is More Informative for Alzheimer’s Disease in Most Frequency Bands

Next, the difference between SCI and AD were assessed when FD metrics were calculated within individual frequency bands. There were no significant differences at any electrode using full time-course estimates of HFD and KFD in the delta (all p > 0.5), alpha (all p > 0.5), beta (all p > 0.1) or gamma (all p > 0.3) bands. In the theta band, no electrodes showed significant differences in full time-course HFD (all p > 0.09).

Within the delta band, mean windowed HFD was significantly higher in the AD group at frontal and parietal electrodes (all t >1.63, p < 0.045). The SD of windowed HFD was not significantly different between groups at any electrodes (all p > 0.1). Similarly, no electrodes showed significant group differences for either Katz FDD metric in the delta band (all p > 0.1). In the theta band, every electrode showed reduced Higuchi FDD measures in AD compared to SCI. Nearly all channels showed significantly lower KFD mean and SD. Finally, mean HFD was significantly higher in the AD group at central and posterior sites, as well as at Fp2, F7, and F8 (all t > 1.64, p < 0.049).

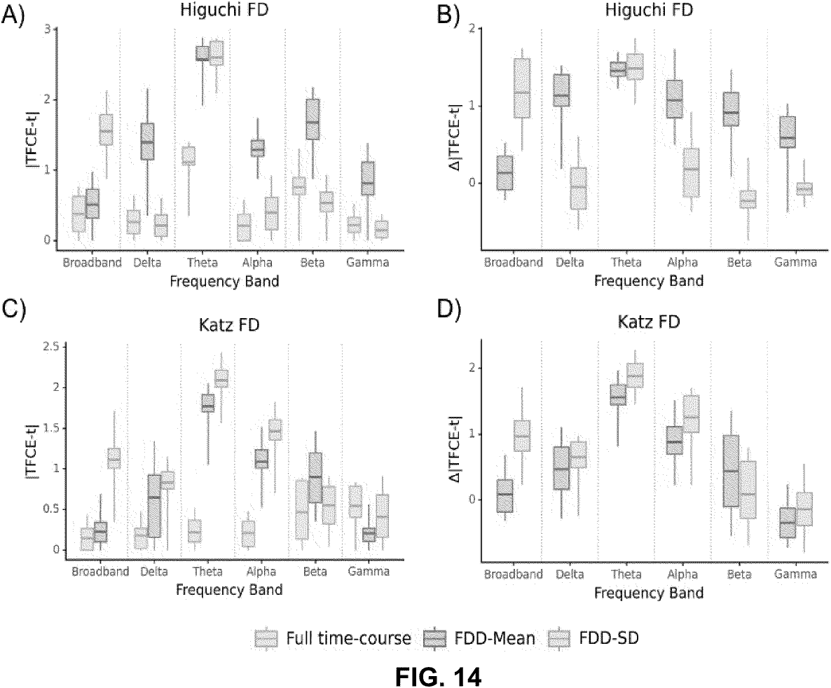

As with the Dem-SCI analysis, he absolute TFCE-t statistics were plotted for AD-SCI at each channel for the full time-course FD compared to TFCE-t scores obtained using FDD measures (FIG. 14), as well as the difference between FDD features and the full time-course FD metrics. The most pronounced improvement was in the theta band. FIG. 14 provides the average channel absolute TFCE-t statistic for AD-SCI in each frequency band using Higuchi (A) or Katz (C). Difference between FDD metrics and full time-course FD in each frequency band (B and D). Whiskers extend to minimum and maximum value.

Averaging across frequency bands, models using FDD had a lower AIC and higher R² than models using full time-course estimates (ΔAIC = -2.6, ΔR² = 0.08). Models using Higuchi FDD were nearly indistinguishable, but had improved class separation relative to full time-course models (Table 5; average ΔAIC = -0.2, ΔR² = 0.08), while models using Katz FDD had lower AIC while showing a minimal increase in class separation. (Table 5; average ΔAIC = -2.6, ΔR² = 0.01).

Models using Higuchi FDD outperformed models using full time-course HFD in the delta (Table 5; ΔAIC = -9.8, ΔR² = 0.05) and alpha bands (ΔAIC = -5.4, ΔR² = 0.30). In the theta and gamma bands, the windowed model separated the classes better, but had worse expected prediction error (ΔAIC = 2.0, ΔR² = 0.09; ΔAIC = 8.9, ΔR² = 0.05). The model using FDD features had lower performance than the models using full time-course HFD in the beta band (ΔAIC = 3.2, ΔR² = -0.09).

The models using KFD were similarly mixed. Models using FDD features outperformed models using full time-course FD in the delta (ΔAIC = -12.1, ΔR² = 0.22) and gamma bands (ΔAIC = -6.8, ΔR² = 0.23), but underperformed models using full time-course FD in the alpha (ΔAIC = 2.2, ΔR² = -0.06) and beta bands (ΔAIC = 4.4, ΔR² = -0.20). In the theta band, models based on FDD had lower AIC but also lower class separation (ΔAIC = -2.0, ΔR² = -0.22).

Across all models comparing AD and SCI, the models with the lowest AIC and largest R² used Higuchi FDD in the delta band (AIC=144.0, R² = 0.342, X²(17)=51.40, p < 0.001) or Katz FDD with broadband EEG (AIC=148.9, R² = 0.507, X²(32)=76.48, p < 0.001) Features useful at predicting AD and dementia partially overlap

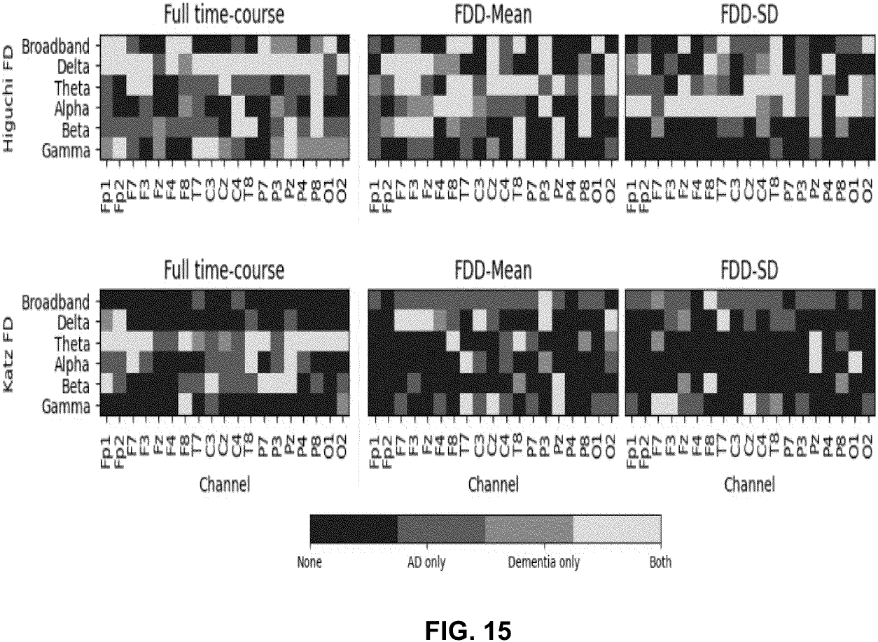

To determine which aspects of windowed FD distributions are most useful for distinguishing between healthy subjects and those with dementia the regression coefficients were examined from each Dementia-SCI and AD-SCI model. In these LASSO regressions, L1 regularization selects variables that relate to the dependent variable, and sets all other coefficients to zero. An examination occurred to determine which channels, frequencies, and FD analysis methods were retained in these models as non-zero coefficients. As shown in FIG. 15, there was partial overlap between useful features identified by the regularized logistic regression. Across the fractal variability models, Dementia-SCI models used a total of 122 EEG features while AD-SCI models used 191, with 89 features appearing in both models.

FIG. 15 provides channels with non-zero coefficients in the regularized logistic regression using full time-course FD (left) or FDD (center and right) features calculated using Higuchi (top) or Katz (bottom) methods.

### V. FDD Provides Information About Dementia Across Different Window Lengths

In order to understand whether the prior results depended on the length of the window used to calculate FDD features, the analysis was repeated using three other window sizes — 0.5 s, 5 s, 10 s — each with a 50% overlap. The method used an estimated ideal kₘₐₓ= 25 for 0.5 s and 5 s windows, and kₘₐₓ = 28 for 10 s windows. Models using FDD continued to generally outperform models using full time-course FD values, with better performance in broadband for both Dementia-SCI and AD-SCI. (see Supplemental Tables S1-S4). Across analyses comparing Dementia-SCI within specific frequency bands, models using FDD features calculated with 1 s windows outperformed models using full time-course FD in 8/10 cases. Similarly, using 0.5 s or 5 s windows resulted in better performance with FDD features in 9/10 cases and FDD calculated from 10 s windows produced better performance in 6/10 cases. When modeling AD-SCI, FDD with 1 s windows outperformed full time-course FD in 5/10 models. Changing the window size resulted in similar performance for 0.5 s (5/10 models), 5 s (4/10 models), or 10 s (6/10) durations.

### Discussion

The first goal of this Example was to investigate the relationship between the fractal dimension distributions and dementia, and to determine whether the distribution of fractal dimension values within windows can outperform traditional approaches that estimate fractal dimension once across a resting-state recording. The results show that FDD carries information above and beyond the full time-course fractal dimension of a signal, and that FDD features are useful at distinguishing individuals diagnosed with dementia from SCI, as well as individuals with AD-dementia from SCI. Using both Higuchi and Katz algorithms, FDD calculated from broadband EEG revealed more significant differences between groups than full time-course fractal values (FIGS. 11 and 12). Models using FDD features were also better (as measured by lower AIC and higher R²) than models using full time-course FD (Tables 5 and 6). FDD features also demonstrated more group differences, larger effect sizes, and better models across most frequency bands (Tables 5 and 6; FIGS. 11 and 12).

A second goal of this Example was to understand whether the distribution of fractal dimension scores could also elucidate characteristics of AD-dementia. FDD features again revealed more electrodes with significant AD-SCI differences than full time-course FD, both in broadband and the majority of traditional frequency bands (FIGS. 13 and 14). Using broadband data, FDD features produced better models for both HFD and KFD (Tables 7 and 8). Models using FDD to predict AD-SCI were better in the delta band and slightly better on average.

Previous work has found lower HFD in AD (Gómez et al., 2009; Gómez & Hornero, 2010; Nobukawa et al., 2019; Smits et al., 2016). Similarly, prior studies have examined FD separately within canonical frequency bands, and find lower KFD and HFD in AD (Al-Nuaimi et al., 2018; Jacob & Gopakumar, 2018; Puri et al., 2022). Using FDD, the methods and systems of the invention replicate this decrease in FD for the dementia and AD-dementia within the theta and alpha bands. Using full time-course FD, the presently disclosed method reveal non-significant trends toward decreases in FD using full time-course broadband HFD and KFD.

Surprisingly, the presently disclosed FDD-based methods provide a non-significant trend where other studies showed a significant decrease. This may be attributed to the present Example comparing AD/Dementia to SCI, whereas other work used healthy older adults recorded in a laboratory setting (Al-Nuaimi et al., 2017, 2018). There may also be an important difference between FD calculated from EEG compared to MEG (Gómez et al., 2009; Gómez & Hornero, 2010). Moreover, computing HFD relies on the kₘₐₓ parameter, and previous investigations of HFD in dementia did not calculate kₘₐₓ using the approach based on time-course length. In contrast, the presently disclosed methods and systems using FDD metrics reliably identify reduced complexity, for both HFD and KFD.

The present results also highlight the importance of distinguishing between dementia caused by AD from non-AD dementia. Particularly when using FDD, the Dementia and SCI groups demonstrate significant differences across nearly the entire scalp (FIG. 11). In contrast, significant differences between AD and SCI were restricted to fewer electrodes, and appeared primarily in the theta band (FIGS. 13 and 14). Moreover, while 73% of the FDD features that were retained in the Dementia-SCI models were also retained in AD-SCI models, less than half of the features used in the AD-SCI models appeared in the Dementia-SCI models (47%). In other words, while some EEG signals are generally useful for detecting cognitive impairment, EEG is also sensitive to additional signals which are uniquely important for detecting AD.

AD is a neurodegenerative disease with a distinct progression of physical and cognitive symptoms. Its pathology is characterized by two features: extracellular deposits of beta-amyloid plaques, and intracellular accumulations of abnormally phosphorylated tau, called neurofibrillary tangles (Cummings, 2004). Patients with Alzheimer’s dementia exhibit widespread amyloid plaques and neurofibrillary tangles throughout the brain, but beta-amyloid and tau start to amass long before severe cognitive symptoms appear (Hanseeuw et al., 2019). One origin of reduced EEG complexity in AD patients may be an abnormal cortical excitation/inhibition ratio (Lauterborn et al., 2021); beta-amyloid is linked to neuronal hypoactivity, and tau is associated with neuronal hyperactivity (Ranasinghe et al., 2021). Decreases in EEG complexity might also arise from general neurodegeneration, with fewer neurons and fewer interactions between neurons (Dauwels et al., 2011).

Researchers investigating FD in EEG might also benefit from examining FDD features, using the presently disclosed methods, as a complement to full time-course FD methods. Previous work has used FD in EEG signals to identify a variety of neuropsychological and neurocognitive conditions. EEG complexity and FD in schizophrenia has been widely investigated, with studies reporting both increased and decreased FD based on symptomatology, age, and medication status (Akar, Kara, Latifoğlu, et al., 2015; Fernández et al., 2013; Raghavendra et al., 2009; Sabeti et al., 2009). In schizophrenia, the FD also has strong predictive power, and it has been used to distinguish individuals with schizophrenia from healthy controls with high accuracy (Goshvarpour & Goshvarpour, 2020). Other work has examined FD in mood and cognitive disorders, with increased FD reported in depression (Akar, Kara, Agambayev, et al., 2015; Bachmann et al., 2018; Čukié et al., 2020), bipolar disorder (Bahrami et al., 2005), and attention deficit hyperactivity disorder (Mohammadi et al., 2016).

One additional potential advantage of FDD over full time-course fractal measures is that it allows for momentary artifacts to be excluded from the data. If artifacts contaminate a portion of an EEG recording, that artifact could bias estimates of FD. A windowed approach like FDD makes it trivially easy to handle events such as jump artifacts or movement artifacts — simply exclude windows with artifacts from the analysis. Thus, FDD offers the potential to recover usable EEG recordings from a broader range of patients.

The present systems and methods using FDD are novel, in part, because they may investigate fractal dimensions within and across frequency bands in resting-state EEG data. The results from this Example extend the work from the prior Examples linking differences in EEG spectral content and fractal dimension to AD and non-AD dementia. In broadband EEG, and within most of the traditional frequency bands, FDD revealed stronger group differences and more informative features than full time-course FD for both dementia and AD-dementia. Moreover, regularized linear regressions using FDD features were better at accounting for differences between unimpaired subjects and subjects with dementia than models using full time-course fractal dimension. Overall, these findings demonstrate that FDD can provide clinically useful information about cognitive status and AD diagnosis from resting-state EEG above and beyond traditional full time-course methods.

TABLE 5

Summary of models predicting Dementia vs SCI using Katz fractal information

| Band | Fractal features | AIC | Pseudo R² | L1 penalty | nₚₐᵣₐₘ | X2 | p |
|---|---|---|---|---|---|---|---|
| Broadband | | | | | | | |
| | Full time-course | 189.2 | 0.058 | 2.15 | 7 | -2.17 | 0.0035 |
| | FDD | 176.5 | 0.299 | 0.25 | 19 | 16.54 | 0.0006 |
| Delta | | | | | | | |
| | Full time-course | 190.8 | 0.087 | 3.7 | 4 | 0.28 | 0.8698 |
| | FDD | 183.0 | 0.261 | 3.55 | 11 | 22.09 | 0.0086 |
| Theta | | | | | | | |
| | Full time-course | 179.6 | 0.398 | 0.6 | 16 | 35.46 | 0.0013 |
| | FDD | 174.8 | 0.358 | 2.4 | 8 | 24.20 | 0.0005 |
| Alpha | | | | | | | |
| | Full time-course | 187.0 | 0.223 | 3.5 | 5 | 6.03 | 0.1100 |
| | FDD | 186.1 | 0.229 | 5.05 | 6 | 8.96 | 0.0622 |
| Beta | | | | | | | |
| | Full time-course | 188.1 | 0.133 | 2.45 | 7 | 8.93 | 0.1120 |
| | FDD | 178.9 | 0.270 | 4.25 | 7 | 18.12 | 0.0028 |
| Gamma | | | | | | | |
| | Full time-course | 189.1 | 0.135 | 2.85 | 4 | 1.99 | 0.3705 |
| | FDD | 194.1 | 0.232 | 4.7 | 9 | 6.98 | 0.4313 |

The number of non-zero parameters in the model (nₚₐᵣₐₘ) including the intercept. The X² statistic and p-value reflect likelihood ratio test compared to a model with age as the only predictor. FDD: Fractal dimension distributions.

TABLE 6

Summary of models predicting AD vs SCI using Higuchi fractal information

| Band | Fractal features | AIC | Pseudo R² | L1 penalty | nₚₐᵣₐₘ | X2 | p |
|---|---|---|---|---|---|---|---|
| Broadband | | | | | | | |
| | Full time-course | 157.0 | 0.204 | 1.45 | 10 | 24.40 | 0.0020 |
| | FDD | 151.6 | 0.331 | 0.85 | 22 | 53.77 | 0.0001 |
| Delta | | | | | | | |
| | Full time-course | 153.8 | 0.291 | 0.05 | 20 | 47.60 | 0.0002 |
| | FDD | 144.0 | 0.342 | 1.80 | 17 | 51.40 | 0.0000 |
| Theta | | | | | | | |
| | Full time-course | 145.6 | 0.400 | 0.40 | 16 | 47.83 | 0.0000 |
| | FDD | 147.5 | 0.487 | 0.15 | 33 | 79.86 | 0.0000 |
| Alpha | | | | | | | |
| | Full time-course | 165.3 | 0.058 | 1.35 | 8 | 12.13 | 0.0592 |
| | FDD | 159.8 | 0.360 | 0.40 | 29 | 59.55 | 0.0003 |
| Beta | | | | | | | |
| | Full time-course | 149.7 | 0.300 | 0.35 | 18 | 47.70 | 0.0001 |
| | FDD | 152.9 | 0.214 | 1.80 | 14 | 36.54 | 0.0003 |
| Gamma | | | | | | | |
| | Full time-course | 156.7 | 0.067 | 1.30 | 8 | 20.74 | 0.0020 |
| | FDD | 165.6 | 0.117 | 2.25 | 11 | 17.84 | 0.0371 |

The number of non-zero parameters in the model (nₚₐᵣₐₘ) including the intercept. The X² statistic and p-value reflect likelihood ratio test compared to a model with age as the only predictor. FDD: Fractal dimension distributions.

TABLE 7

Summary of models predicting AD vs SCI using Katz fractal information

| Band | Fractal features | AIC | Pseudo R² | L1 penalty | nₚₐᵣₐₘ | X2 | p |
|---|---|---|---|---|---|---|---|
| Broadband | | | | | | | |
| | Full time-course | 159.6 | 0.088 | 2.90 | 4 | 9.77 | 0.0076 |
| | FDD | 148.9 | 0.507 | 0.30 | 32 | 76.48 | 0.0000 |
| Delta | | | | | | | |
| | Full time-course | 167.6 | 0.009 | 2.45 | 5 | 3.83 | 0.2804 |
| | FDD | 155.5 | 0.224 | 2.15 | 15 | 35.90 | 0.0006 |
| Theta | | | | | | | |
| | Full time-course | 156.8 | 0.341 | 0.15 | 19 | 42.64 | 0.0005 |
| | FDD | 154.8 | 0.126 | 2.25 | 8 | 22.64 | 0.0009 |
| Alpha | | | | | | | |
| | Full time-course | 162.7 | 0.165 | 1.25 | 13 | 24.68 | 0.0101 |
| | FDD | 164.9 | 0.107 | 2.75 | 8 | 12.49 | 0.0519 |
| Beta | | | | | | | |
| | Full time-course | 159.1 | 0.252 | 0.65 | 15 | 32.26 | 0.0022 |
| | FDD | 163.6 | 0.049 | 4.45 | 5 | 7.83 | 0.0496 |
| Gamma | | | | | | | |
| | Full time-course | 168.5 | 0.000 | 3.80 | 4 | 0.93 | 0.6266 |
| | FDD | 161.7 | 0.233 | 1.20 | 21 | 41.69 | 0.0020 |

The number of non-zero parameters in the model (nₚₐᵣₐₘ) including the intercept. The X² statistic and p-value reflect likelihood ratio test compared to a model with age as the only predictor. FDD: Fractal dimension distributions.

TABLE S1

Summary of models predicting Dementia vs SCI using Higuchi fractal information with different window lengths

| Band | Model | | Window AIC | Pseudo R² | L1 penalty | k | X2 | p |
|---|---|---|---|---|---|---|---|---|
| Broadband | | | | | | | | |
| | Full time-course HFD | | 180.3 | 0.290 | 1.6 | 11 | 24.74 | 0.0033 |
| | FDD features | 0.5 s | 171.6 | 0.310 | 3.75 | 5 | 21.41 | 0.0001 |
| | FDD features | 1 s | 163.6 | 0.446 | 1.85 | 6 | 47.40 | 0.0111 |
| | FDD features | 5 s | 171.7 | 0.339 | 4.1 | 6 | 23.31 | 0.0001 |
| | FDD features | 10 s | 173.0 | 0.339 | 3.4 | 7 | 24.07 | 0.0002 |
| Delta | | | | | | | | |
| | Full time-course HFD | | 178.8 | 0.425 | 0.25 | 19 | 42.20 | 0.0006 |
| | FDD features | 0.5 s | 182.3 | 0.252 | 0.65 | 11 | 22.70 | 0.0069 |
| | FDD features | 1 s | 176.1 | 0.397 | 1.9 | 19 | 44.91 | 0.0003 |
| | FDD features | 5 s | 178.7 | 0.371 | 3 | 10 | 24.32 | 0.0020 |
| | FDD features | 10 s | 180.9 | 0.409 | 1.75 | 13 | 28.17 | 0.0030 |
| Theta | | | | | | | | |
| | Full time-course HFD | | 174.9 | 0.269 | 1.7 | 6 | 20.13 | 0.0005 |
| | FDD features | 0.5 s | 164.1 | 0.582 | 0.1 | 34 | 86.91 | 0.0000 |
| | FDD features | 1 s | 159.6 | 0.592 | 0.85 | 23 | 69.44 | 0.0000 |
| | FDD features | 5 s | 165.4 | 0.494 | 2.25 | 9 | 35.61 | 0.0000 |
| | FDD features | 10 s | 176.3 | 0.259 | 3.25 | 6 | 18.79 | 0.0009 |
| Alpha | | | | | | | | |
| | Full time-course HFD | | 182.0 | 0.262 | 1.85 | 6 | 13.04 | 0.0111 |
| | FDD features | 0.5 s | 171.0 | 0.467 | 0.65 | 20 | 52.08 | 0.0000 |
| | FDD features | 1 s | 166.6 | 0.644 | 0.75 | 25 | 66.42 | 0.0000 |
| | FDD features | 5 s | 170.0 | 0.476 | 0.9 | 23 | 59.06 | 0.0000 |
| | FDD features | 10 s | 173.8 | 0.507 | 0.95 | 21 | 51.25 | 0.0001 |
| Beta | | | | | | | | |
| | Full time-course HFD | | 179.5 | 0.240 | 2.15 | 7 | 17.57 | 0.0035 |
| | FDD features | 0.5 s | 171.0 | 0.349 | 1.05 | 12 | 36.06 | 0.0001 |
| | FDD features | 1 s | 173.0 | 0.377 | 1.9 | 14 | 38.05 | 0.0002 |
| | FDD features | 5 s | 169.2 | 0.407 | 1 | 20 | 53.84 | 0.0000 |
| | FDD features | 10 s | 175.4 | 0.565 | 0.45 | 28 | 63.63 | 0.0001 |
| Gamma | | | | | | | | |
| | Full time-course HFD | | 184.4 | 0.298 | 0.6 | 14 | 26.65 | 0.0087 |
| | FDD features | 0.5 s | 176.3 | 0.389 | 0.25 | 16 | 38.77 | 0.0004 |
| | FDD features | 1 s | 184.7 | 0.220 | 4.75 | 5 | 8.33 | 0.0396 |
| | FDD features | 5 s | 182.3 | 0.279 | 1.6 | 12 | 24.77 | 0.0058 |
| | FDD features | 10 s | 187.2 | 0.096 | 4.1 | 3 | 1.85 | 0.1742 |

The number of non-zero parameters in the model (k) including the intercept. HFD: Higuchi fractal dimension.

TABLEs S2

Summary of models predicting Dementia vs SCI using Katz fractal information

| Band | Model | Window | AIC | Pseudo R² | L1 penalty | k | X2 | p |
|---|---|---|---|---|---|---|---|---|
| Broadband | | | | | | | | |
| | Full time-course KFD | | 189.2 | 0.058 | 5.65 | 2 | -2.17 | 1.0000 |
| | FDD features | 0.5 s | 175.6 | 0.269 | 4.65 | 6 | 19.48 | 0.0006 |
| | FDD features | 1 s | 176.5 | 0.299 | 0.25 | 19 | 16.54 | 0.0006 |
| | FDD features | 5 s | 182.0 | 0.152 | 4.9 | 5 | 11.05 | 0.0115 |
| | FDD features | 10 s | 185.4 | 0.153 | 4.15 | 6 | 9.64 | 0.0470 |
| Delta | | | | | | | | |
| | Full time-course KFD | | 190.8 | 0.087 | 3.7 | 4 | 0.28 | 0.8698 |
| | FDD features | 0.5 s | 184.0 | 0.476 | 1.25 | 24 | 47.09 | 0.0014 |
| | FDD features | 1 s | 183.0 | 0.261 | 3.55 | 11 | 22.09 | 0.0086 |
| | FDD features | 5 s | 187.3 | 0.486 | 0.7 | 28 | 51.76 | 0.0019 |
| | FDD features | 10 s | 189.6 | 0.202 | 4.75 | 6 | 5.45 | 0.2440 |
| Theta | | | | | | | | |
| | Full time-course KFD | | 179.6 | 0.398 | 0.6 | 16 | 35.46 | 0.0013 |
| | FDD features | 0.5 s | 165.0 | 0.592 | 0.45 | 28 | 74.03 | 0.0000 |
| | FDD features | 1 s | 174.8 | 0.358 | 2.4 | 8 | 24.20 | 0.0005 |
| | FDD features | 5 s | 167.8 | 0.653 | 0.85 | 26 | 67.27 | 0.0000 |
| | FDD features | 10 s | 175.6 | 0.603 | 0.8 | 27 | 61.45 | 0.0001 |
| Alpha | | | | | | | | |
| | Full time-course KFD | | 187.0 | 0.223 | 3.5 | 5 | 6.03 | 0.1100 |
| | FDD features | 0.5 s | 185.0 | 0.211 | 5.4 | 5 | 8.04 | 0.0451 |
| | FDD features | 1 s | 186.1 | 0.229 | 5.05 | 6 | 8.96 | 0.0622 |
| | FDD features | 5 s | 190.2 | 0.125 | 4.5 | 5 | 2.85 | 0.4160 |
| | FDD features | 10 s | 190.6 | 0.058 | 5.1 | 3 | -1.52 | 1.0000 |
| Beta | | | | | | | | |
| | Full time-course KFD | | 188.1 | 0.133 | 2.45 | 7 | 8.93 | 0.1120 |
| | FDD features | 0.5 s | 186.6 | 0.280 | 3.05 | 11 | 18.40 | 0.0308 |
| | FDD features | 1 s | 178.9 | 0.270 | 4.25 | 7 | 18.12 | 0.0028 |
| | FDD features | 5 s | 177.0 | 0.429 | 2.65 | 12 | 30.05 | 0.0008 |
| | FDD features | 10 s | 182.0 | 0.302 | 2.8 | 11 | 23.00 | 0.0062 |
| Gamma | | | | | | | | |
| | Full time-course KFD | | 189.1 | 0.135 | 2.85 | 4 | 1.99 | 0.3705 |
| | FDD features | 0.5 s | 186.9 | 0.329 | 2.4 | 12 | 20.17 | 0.0277 |
| | FDD features | 1 s | 194.1 | 0.232 | 4.7 | 9 | 6.98 | 0.4313 |
| | FDD features | 5 s | 185.9 | 0.145 | 4.65 | 4 | 5.13 | 0.0768 |
| | FDD features | 10 s | 189.0 | 0.036 | 4.35 | 4 | 2.04 | 0.3605 |

The number of non-zero parameters in the model (k) including the intercept. KFD: Katz fractal dimension.

TABLE S3

Summary of models predicting Dementia vs SCI using Higuchi fractal information with different window lengths

| Band | Model | Window | AIC | Pseudo R² | L1 penalty | k | X2 | p |
|---|---|---|---|---|---|---|---|---|
| Broadband | | | | | | | | |
| | Full time-course | | | | | | | |
| | HFD | | 157.0 | 0.204 | 1.45 | 10 | 24.40 | 0.0020 |
| | FDD features | 0.5 s | 151.1 | 0.341 | 0.6 | 25 | 60.24 | 0.0000 |
| | FDD features | 1 s | 151.6 | 0.331 | 0.85 | 22 | 53.77 | 0.0001 |
| | FDD features | 5 s | 152.5 | 0.380 | 0.55 | 27 | 62.88 | 0.0000 |
| | FDD features | 10 s | 150.2 | 0.292 | 0.9 | 21 | 53.23 | 0.0000 |
| Delta | | | | | | | | |
| | Full time-course | | | | | | | |
| | HFD | | 153.8 | 0.291 | 0.05 | 20 | 47.60 | 0.0002 |
| | FDD features | 0.5 s | 155.1 | 0.253 | 0.25 | 18 | 42.30 | 0.0004 |
| | FDD features | 1 s | 144.0 | 0.342 | 1.80 | 17 | 51.40 | 0.0000 |
| | FDD features | 5 s | 156.4 | 0.254 | 2.6 | 12 | 28.98 | 0.0013 |
| | FDD features | 10 s | 159.9 | 0.117 | 3 | 9 | 19.47 | 0.0068 |

Theta

| | Full time-course | | | | | | | |
|---|---|---|---|---|---|---|---|---|
| | HFD | | 145.6 | 0.400 | 0.40 | 16 | 47.83 | 0.0000 |
| | FDD features | 0.5 s | 155.6 | 0.185 | 1.6 | 9 | 23.82 | 0.0012 |
| | FDD features | 1 s | 147.5 | 0.487 | 0.15 | 33 | 79.86 | 0.0000 |
| | FDD features | 5 s | 154.6 | 0.205 | 2.5 | 9 | 24.84 | 0.0008 |
| | FDD features | 10 s | 159.8 | 0.068 | 3.45 | 6 | 13.57 | 0.0088 |
| Alpha | | | | | | | | |
| | Full time-course | | | | | | | |
| | HFD | | 165.3 | 0.058 | 1.35 | 8 | 12.13 | 0.0592 |
| | FDD features | 0.5 s | 160.4 | 0.156 | 1.15 | 11 | 22.94 | 0.0063 |
| | FDD features | 1 s | 159.8 | 0.360 | 0.40 | 29 | 59.55 | 0.0003 |
| | FDD features | 5 s | 165.7 | 0.039 | 4.15 | 5 | 5.69 | 0.1279 |
| | FDD features | 10 s | 158.6 | 0.439 | 0.25 | 31 | 64.77 | 0.0002 |
| Beta | | | | | | | | |
| | Full time-course | | | | | | | |
| | HFD | | 149.7 | 0.300 | 0.35 | 18 | 47.70 | 0.0001 |
| | FDD features | 0.5 s | 160.5 | 0.459 | 0.15 | 34 | 68.92 | 0.0002 |
| | FDD features | 1 s | 152.9 | 0.214 | 1.80 | 14 | 36.54 | 0.0003 |
| | FDD features | 5 s | 149.7 | 0.547 | 0.1 | 37 | 85.73 | 0.0000 |
| | FDD features | 10 s | 166.0 | 0.059 | 3.85 | 6 | 7.42 | 0.1155 |
| Gamma | | | | | | | | |
| | Full time-course | | | | | | | |
| | HFD | | 156.7 | 0.067 | 1.30 | 8 | 20.74 | 0.0020 |
| | FDD features | 0.5 s | 157.0 | 0.107 | 0.55 | 10 | 24.39 | 0.0020 |
| | FDD features | 1 s | 165.6 | 0.117 | 2.25 | 11 | 17.84 | 0.0371 |
| | FDD features | 5 s | 164.7 | 0.175 | 1.55 | 14 | 24.73 | 0.0161 |
| | FDD features | 10 s | 166.7 | 0.020 | 3.6 | 6 | 6.68 | 0.1537 |

The number of non-zero parameters in the model (k) including the intercept. HFD: Higuchi fractal dimension.

TABLE S4

Summary of models predicting Dementia vs SCI using Katz fractal information with different window lengths

| Band | Model | Window | AIC | Pseudo R² | L1 penalty | k | X2 | p |
|---|---|---|---|---|---|---|---|---|
| Broadband | | | | | | | | |
| | Full time-course KFD | | 159.6 | 0.088 | 2.90 | 4 | 9.77 | 0.0076 |
| | FDD features | 0.5 s | 155.1 | 0.301 | 1.1 | 22 | 50.29 | 0.0002 |
| | FDD features | 1 s | 148.9 | 0.507 | 0.30 | 32 | 76.48 | 0.0000 |
| | FDD features | 5 s | 156.2 | 0.213 | 1.1 | 18 | 41.15 | 0.0005 |
| | FDD features | 10 s | 158.1 | 0.322 | 0.75 | 22 | 47.24 | 0.0005 |
| | Full time-course | | | | | | | |
| Delta | | | | | | | | |
| | Full time-course KFD | | 167.6 | 0.009 | 2.45 | 5 | 3.83 | 0.2804 |
| | FDD features | 0.5 s | 150.6 | 0.262 | 1.95 | 14 | 38.82 | 0.0001 |
| | FDD features | 1 s | 155.5 | 0.224 | 2.15 | 15 | 35.90 | 0.0006 |
| | FDD features | 5 s | 167.9 | 0.117 | 2.8 | 14 | 21.49 | 0.0436 |
| | FDD features | 10 s | 162.3 | 0.283 | 1.6 | 20 | 39.14 | 0.0027 |
| Theta | | | | | | | | |
| | Full time-course KFD | | 156.8 | 0.341 | 0.15 | 19 | 42.64 | 0.0005 |
| | FDD features | 0.5 s | 154.4 | 0.234 | 1.5 | 14 | 35.00 | 0.0005 |
| | FDD features | 1 s | 154.8 | 0.126 | 2.25 | 8 | 22.64 | 0.0009 |
| | FDD features | 5 s | 150.9 | 0.215 | 2.4 | 10 | 30.49 | 0.0002 |
| | FDD features | 10 s | 151.5 | 0.263 | 1.85 | 12 | 33.88 | 0.0002 |
| Alpha | | | | | | | | |
| | Full time-course KFD | | 162.7 | 0.165 | 1.25 | 13 | 24.68 | 0.0101 |
| | FDD features | 0.5 s | 162.6 | 0.020 | 5.25 | 3 | 4.78 | 0.0289 |
| | FDD features | 1 s | 164.9 | 0.107 | 2.75 | 8 | 12.49 | 0.0519 |
| | FDD features | 5 s | 171.0 | 0.020 | 2.65 | 10 | 10.35 | 0.2410 |
| | FDD features | 10 s | 166.2 | 0.000 | 3.4 | 5 | 5.18 | 0.1589 |
| Beta | | | | | | | | |
| | Full time-course KFD | | 159.1 | 0.252 | 0.65 | 15 | 32.26 | 0.0022 |
| | FDD features | 0.5 s | 161.9 | 0.429 | 0.15 | 33 | 65.54 | 0.0003 |
| | FDD features | 1 s | 163.6 | 0.049 | 4.45 | 5 | 7.83 | 0.0496 |
| | FDD features | 5 s | 148.8 | 0.330 | 0.85 | 26 | 64.59 | 0.0000 |
| | FDD features | 10 s | 145.8 | 0.272 | 2.05 | 14 | 43.59 | 0.0000 |
| Gamma | | | | | | | | |
| | Full time-course KFD | | 168.5 | 0.000 | 3.80 | 4 | 0.93 | 0.6266 |
| | FDD features | 0.5 s | 164.1 | 0.147 | 2.8 | 11 | 19.29 | 0.0228 |
| | FDD features | 1 s | 161.7 | 0.233 | 1.20 | 21 | 41.69 | 0.0020 |
| | FDD features | 5 s | 165.9 | 0.058 | 2.85 | 13 | 21.51 | 0.0285 |
| | FDD features | 10 s | 168.2 | 0.029 | 3.8 | 8 | 9.20 | 0.1628 |

The number of non-zero parameters in the model (k) including the intercept. KFD: Katz fractal dimension.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

### REFERENCES

Accardo, A., Affinito, M., Carrozzi, M., & Bouquet, F. (1997). Use of the fractal dimension for the analysis of electroencephalographic time series. Biological Cybernetics, 77(5), 339-350. Ahmadlou, M., Adeli, H., & Adeli, A. (2011). Fractality and a Wavelet-chaos-Methodology for EEG-based Diagnosis of Alzheimer Disease. Alzheimer Disease & Associated Disorders, 25(1), 85-92.

Akar, S. A., Kara, S., Agambayev, S., & Bilgiç, V. (2015). Nonlinear analysis of EEGs of patients with major depression during different emotional states. Computers in Biology and Medicine, 67, 49-60.

Akar, S. A., Kara, S., Latifoğlu, F., & Bilgiç, V. (2015). Investigation of the noise effect on fractal dimension of EEG in schizophrenia patients using wavelet and SSA-based approaches. Biomedical Signal Processing and Control, 18, 42-48.

Al-Nuaimi, A. H. H., Jammeh, E., Sun, L., & Ifeachor, E. (2017). Higuchi fractal dimension of the electroencephalogram as a biomarker for early detection of Alzheimer’s disease. 2017 39th Annual International Conference of the IEEE Engineering in Medicine and Biology Society (EMBC), 2320-2324.

Al-Nuaimi, A. H. H., Jammeh, E., Sun, L., & Ifeachor, E. (2018). Complexity Measures for Quantifying Changes in Electroencephalogram in Alzheimer’s Disease. Complexity, 2018, 1-12. Amezquita-Sanchez, J. P., Mammone, N., Morabito, F. C., Marino, S., & Adeli, H. (2019). A novel methodology for automated differential diagnosis of mild cognitive impairment and the Alzheimer’s disease using EEG signals. Journal of Neuroscience Methods, 322, 88-95. Arevalo-Rodriguez, I., Smailagic, N., Figuls, M. R. i, Ciapponi, A., Sanchez-Perez, E., Giannakou, A., Pedraza, O. L., Cosp, X. B., & Cullum, S. (2015). Mini-Mental State Examination (MMSE) for the detection of Alzheimer’s disease and other dementias in people with mild cognitive impairment (MCI). Cochrane Database of Systematic Reviews, 3. Bachmann, M., Päeske, L., Kalev, K., Aarma, K., Lehtmets, A., Ööpik, P., Lass, J., & Hinrikus, H. (2018). Methods for classifying depression in single channel EEG using linear and nonlinear signal analysis. Computer Methods and Programs in Biomedicine, 155, 11-17.

Bahrami, B., Seyedsadjadi, R., Babadi, B., & Noroozian, M. (2005). Brain complexity increases in mania. NeuroReport, 16(2), 187-191.

Bergeron, D., Flynn, K., Verret, L., Poulin, S., Bouchard, R. W., Bocti, C., Fülöp, T., Lacombe, G., Gauthier, S., Nasreddine, Z., & Laforce, R. J. (2017). Multicenter Validation of an MMSE-MoCA Conversion Table. Journal of the American Geriatrics Society, 65(5), 1067-1072.

Čukić, M., Stokić, M., Radenković, S., Ljubisavljević, M., Simić, S., & Savić, D. (2020). Nonlinear analysis of EEG complexity in episode and remission phase of recurrent depression. International Journal of Methods in Psychiatric Research, 29(2), e1816.

Cummings, J. L. (2004). Alzheimer’s Disease. New England Journal of Medicine, 351(1), 56-67.

Dauwels, J., Srinivasan, K., Ramasubba Reddy, M., Musha, T., Vialatte, F.-B., Latchoumane, C., Jeong, J., & Cichocki, A. (2011). Slowing and Loss of Complexity in Alzheimer’s EEG: Two Sides of the Same Coin? International Journal of Alzheimer’s Disease, 2011, 539621.

Doyle, T. L. A., Dugan, E. L., Humphries, B., & Newton, R. U. (2004). Discriminating between elderly and young using a fractal dimension analysis of centre of pressure. International Journal of Medical Sciences, 1(1), 11-20.

Fernández, A., Gómez, C., Hornero, R., & López-Ibor, J. J. (2013). Complexity and schizophrenia. Progress in Neuro-Psychopharmacology and Biological Psychiatry, 45, 267-276. Folstein, M. F., Folstein, S. E., & McHugh, P. R. (1975). “Mini-mental state”. A practical method for grading the cognitive state of patients for the clinician. Journal of Psychiatric Research, 12(3), 189-198.

Gómez, C., & Hornero, R. (2010). Entropy and Complexity Analyses in Alzheimer’s Disease: An MEG Study. The Open Biomedical Engineering Journal, 4(1), 223-235.

Gómez, C., Mediavilla, Á., Hornero, R., Abásolo, D., & Fernández, A. (2009). Use of the Higuchi’s fractal dimension for the analysis of MEG recordings from Alzheimer’s disease patients. Medical Engineering & Physics, 31(3), 306-313.

Goshvarpour, A., & Goshvarpour, A. (2020). Schizophrenia diagnosis using innovative EEG feature-level fusion schemes. Physical and Engineering Sciences in Medicine, 43(1), 227-238. Hanseeuw, B. J., Betensky, R. A., Jacobs, H. I. L., Schultz, A. P., Sepulcre, J., Becker, J. A., Cosio, D. M. O., Farrell, M., Quiroz, Y. T., Mormino, E. C., Buckley, R. F., Papp, K. V., Amariglio, R. A., Dewachter, I., Ivanoiu, A., Huijbers, W., Hedden, T., Marshall, G. A., Chhatwal, J. P., ... Johnson, K. (2019). Association of Amyloid and Tau With Cognition in Preclinical Alzheimer Disease: A Longitudinal Study. JAMA Neurology, 76(8), 915-924. Hebert, L. E., Beckett, L. A., Scherr, P. A., & Evans, D. A. (2001). Annual Incidence of Alzheimer Disease in the United States Projected to the Years 2000 Through 2050. Alzheimer Disease & Associated Disorders, 15(4), 169-173.

Higuchi, T. (1988). Approach to an irregular time series on the basis of the fractal theory. Physica D: Nonlinear Phenomena, 31(2), 277-283.

Jacob, J. E., & Gopakumar, K. (2018). Automated Diagnosis of Encephalopathy Using Fractal Dimensions of EEG Sub-Bands. 2018 IEEE Recent Advances in Intelligent Computational Systems (RAICS), 94-97.

Langa, K. M., & Levine, D. A. (2014). The Diagnosis and Management of Mild Cognitive Impairment: A Clinical Review. JAMA, 312(23), 2551-2561.

Lau, Z. J., Pham, T., Chen, S. H. A., & Makowski, D. (2022). Brain entropy, fractal dimensions and predictability: A review of complexity measures for EEG in healthy and neuropsychiatric populations. European Journal of Neuroscience, 56 Lauterborn, J. C., Scaduto, P., Cox, C. D., Schulmann, A., Lynch, G., Gall, C. M., Keene, C. D., & Limon, A. (2021). Increased excitatory to inhibitory synaptic ratio in parietal cortex samples from individuals with Alzheimer’s disease. Nature Communications, 12(1), Article 1.

McKhann, G. M., Knopman, D. S., Chertkow, H., Hyman, B. T., Jack, C. R., Kawas, C. H., Klunk, W. E., Koroshetz, W. J., Manly, J. J., Mayeux, R., Mohs, R. C., Morris, J. C., Rossor, M. N., Scheltens, P., Carrillo, M. C., Thies, B., Weintraub, S., & Phelps, C. H. (2011). The diagnosis of dementia due to Alzheimer’s disease: Recommendations from the National Institute on Aging-Alzheimer’s Association workgroups on diagnostic guidelines for Alzheimer’s disease. Alzheimer’s & Dementia, 7(3), 263-269.

Mensen, A., & Khatami, R. (2013). Advanced EEG analysis using threshold-free cluster-enhancement and non-parametric statistics. NeuroImage, 67, 111-118.

Mohammadi, M. R., Khaleghi, A., Nasrabadi, A. M., Rafieivand, S., Begol, M., & Zarafshan, H. (2016). EEG classification of ADHD and normal children using non-linear features and neural network. Biomedical Engineering Letters, 6(2), 66-73.

Nasreddine, Z. S., Phillips, N. A., Bédirian, V., Charbonneau, S., Whitehead, V., Collin, I., Cummings, J. L., & Chertkow, H. (2005). The Montreal Cognitive Assessment, MoCA: A Brief Screening Tool For Mild Cognitive Impairment. Journal of the American Geriatrics Society, 53(4), 695-699.

Nobukawa, S., Yamanishi, T., Nishimura, H., Wada, Y., Kikuchi, M., & Takahashi, T. (2019). Atypical temporal-scale-specific fractal changes in Alzheimer’s disease EEG and their relevance to cognitive decline. Cognitive Neurodynamics, 13(1), 1-11.

Puri, D. V., Nalbalwar, S., Nandgaonkar, A., & Wagh, A. (2022). Alzheimer’s disease detection from optimal EEG channels and Tunable Q-Wavelet Transform. Indonesian Journal of Electrical Engineering and Computer Science, 25(3), 1420.

Raghavendra, B. S., Dutt, D. N., Halahalli, H. N., & John, J. P. (2009). Complexity analysis of EEG in patients with schizophrenia using fractal dimension. Physiological Measurement, 30(8), 795-808.

Rajan, K. B., Weuve, J., Barnes, L. L., McAninch, E. A., Wilson, R. S., & Evans, D. A. (2021). Population estimate of people with clinical Alzheimer’s disease and mild cognitive impairment in the United States (2020-2060). Alzheimer’s & Dementia, 17(12), 1966-1975.

Ranasinghe, K. G., Verma, P., Cai, C., Xie, X., Kudo, K., Gao, X., Lerner, H. M., Mizuiri, D., Strom, A., Iaccarino, L., La Joie, R., Miller, B. L., Tempini, M. L. G., Rankin, K. P., Jagust, W. J., Vossel, K. A., Rabinovici, G. D., Raj, A., & Nagaraj an, S. S. (2021). Abnormal neural oscillations depicting excitatory-inhibitory imbalance are distinctly associated with amyloid and tau depositions in Alzheimer’s disease. Alzheimer’s & Dementia, 17(S4).

Reisberg, B., Prichep, L., Mosconi, L., John, E. R., Glodzik-Sobanska, L., Boksay, I., Monteiro, I., Torossian, C., Vedvyas, A., Ashraf, N., Jamil, I. A., & de Leon, M. J. (2008). The pre-mild cognitive impairment, subjective cognitive impairment stage of Alzheimer’s disease. Alzheimer’s & Dementia, 4(1, Supplement 1), S98-S108.

Rossini, P. M., Di Iorio, R., Vecchio, F., Anfossi, M., Babiloni, C., Bozzali, M., Bruni, A. C., Cappa, S. F., Escudero, J., Fraga, F. J., Giannakopoulos, P., Guntekin, B., Logroscino, G., Marra, C., Miraglia, F., Panza, F., Tecchio, F., Pascual-Leone, A., & Dubois, B. (2020). Early diagnosis of Alzheimer’s disease: The role of biomarkers including advanced EEG signal analysis. Report from the IFCN-sponsored panel of experts. Clinical Neurophysiology, 131(6), 1287-1310. Sabeti, M., Katebi, S., & Boostani, R. (2009). Entropy and complexity measures for EEG signal classification of schizophrenic and control participants. Artificial Intelligence in Medicine, 47(3), 263-274.

Schmand, B., Eikelenboom, P., van Gool, W. A., & Initiative, the A. D. N. (2011). Value of Neuropsychological Tests, Neuroimaging, and Biomarkers for Diagnosing Alzheimer’s Disease in Younger and Older Age Cohorts. Journal of the American Geriatrics Society, 59(9), 1705-1710.

Smith, S. M., & Nichols, T. E. (2009). Threshold-free cluster enhancement: Addressing problems of smoothing, threshold dependence and localisation in cluster inference. NeuroImage, 44(1), 83-98.

Smits, F. M., Porcaro, C., Cottone, C., Cancelli, A., Rossini, P. M., & Tecchio, F. (2016). Electroencephalographic Fractal Dimension in Healthy Ageing and Alzheimer’s Disease. PLOS ONE, 11(2), e0149587.

Staudinger, T., & Polikar, R. (2011). Analysis of complexity based EEG features for the diagnosis of Alzheimer’s disease. 2011 Annual International Conference of the IEEE Engineering in Medicine and Biology Society, 2033-2036.

Sun, J., Wang, B., Niu, Y., Tan, Y., Fan, C., Zhang, N., Xue, J., Wei, J., & Xiang, J. (2020). Complexity Analysis of EEG, MEG, and fMRI in Mild Cognitive Impairment and Alzheimer’s Disease: A Review. Entropy, 22(2), Article 2.

Taylor, C. A., Greenlund, S. F., McGuire, L. C., Lu, H., & Croft, J. B. (2017). Deaths from Alzheimer’s Disease-United States, 1999-2014. Morbidity and Mortality Weekly Report, 66(20), 521-526.

Tjur, T. (2009). Coefficients of Determination in Logistic Regression Models—A New Proposal: The Coefficient of Discrimination. The American Statistician, 63(4), 366-372.

Vaz, M., & Silvestre, S. (2020). Alzheimer’s disease: Recent treatment strategies. European Journal of Pharmacology, 887, 173554.

Wajnsztejn, R., Carvalho, T. D. de, Garner*, D. M., Raimundo, R. D., Vanderlei, L. C. M., Godoy, M. F., Ferreira, C., Valenti, V. E., & Abreu, L. C. de. (2016). Higuchi fractal dimension applied to RR intervals in children with Attention Deficit Hyperactivity Disorder. Journal of Human Growth and Development, 26(2), 147-153.

Wanliss, J. A., & Wanliss, G. E. (2022). Efficient calculation of fractal properties via the Higuchi method. Nonlinear Dynamics, 109(4), 2893-2904.

Yang, A. C., Wang, S.-J., Lai, K.-L., Tsai, C.-F., Yang, C.-H., Hwang, J.-P., Lo, M.-T., Huang, N. E., Peng, C.-K., & Fuh, J.-L. (2013). Cognitive and neuropsychiatric correlates of EEG dynamic complexity in patients with Alzheimer’s disease. Progress in Neuro-Psychopharmacology and Biological Psychiatry, 47, 52-61.

1. A system for assessing an Alzheimer’s Disease (AD) status of a subject, the system comprising:
a central processing unit (CPU); and storage coupled to said CPU for storing instructions that when executed by the CPU cause the CPU to:
accept as an input, neural data from a subject, recorded using EEG sensors;
transform the neural data into a time series;
bandpass filter the time series to produce a plurality of different frequency band time series;
repeatedly advance a sliding-window along each frequency band time series and obtain at least one fractal measurement to produce at least one measure time series for each frequency band time series;
extract measures from each measure time series;
combine the extracted measures and analyze the combined measures using a machine learning system trained to correlate features in the combined measures with Alzheimer’s disease to produce at least one Fractal Dimensions Distribution (FDD) score; and
provide an output of the subject’s AD status based on the FDD score.

2. The system of claim 1, wherein producing the FDD score comprises determining summary statistics for the distribution of the fractal measurements.

3. The system of claim 2, wherein the summary statistics comprise one or more of a standard deviation of the fractal measurements, mean of the fractal measurements, skewness of the fractal measurements, and kurtosis of the fractal measurements.

4. The system of claim 1, wherein the extracted measures comprise one or more of:
frequency and/or time frequency measures;
oscillatory measures;
amplitude modulation measures;
spectral connectivity measures;
network analysis measures;
chaos measures;
complexity measures; and
entropy measures.

5. The system of claim 1, wherein the output comprises a continuous score indicating the probability that a subject has Alzheimer’s disease; and:
wherein the system allocates the continuous score into one of a plurality of bins, wherein each bin corresponds to a subject’s risk of developing Alzheimer’s disease; and/or
wherein when the probability drops below a threshold, the output provides indicates that the subject does not have Alzheimer’s disease.

6-7. (canceled)

8. The system of claim 1, wherein the output includes an identification of the extracted measures analyzed by the machine learning system and/or the FDD scores used to provide the output, and optionally the extracted measures provided in the output are identified using a pictorial representation of the analyzed measure.

9. (canceled)

10. The system of claim 1, wherein the neural data is collected from the subject over at least one period of time during which the subject is at rest, and:
wherein the neural data is collected from the subject from at least two periods of time;
wherein the system provides an output of the subject’s AD status after every period of time during which neural data is recorded from the subject; and/or
wherein results from each output are combined to produce a longitudinal assessment of the subject’s AD status.

11. The system of claim 1, wherein the recorded neural data includes one or more annotations identifying one or more of the subject’s age, sex, medical history, results from one or more biomolecular assay, and/or subjective cognitive assessment, and wherein the annotations are provided to the machine learning system for analysis with the combined extracted measures.

12-17. (canceled)

18. The system of claim 1, wherein the fractal measurement is a calculated a Katz fractal dimension (KFD).

19. The system of claim 1, wherein the fractal measurement is a calculated Higuchi fractal dimension (HFD), wherein the calculated HFD is calculated using a kmax estimated as optimal based on a duration of the at least one measure time series.

20. (canceled)

21. A method for assessing an AD status of a subject, the method comprising:
recording using EEG sensors neural data from a subject using EEG sensors;
transforming the neural data into a time series;
filtering the time series to produce a plurality of different frequency band time series;
producing at least one measure time series for each frequency band time series by repeatedly advancing a sliding-window along each frequency band time series and obtaining at least one fractal measurement;
extracting measures from each measure time series;
combining the extracted measure and analyzing the combined measures using a machine learning system trained to correlate features in the combined measures with Alzheimer’s disease to produce at least one fractal dimension distribution (FDD) score; and providing an output of the subject’s AD status based on the FDD score.

22. The method of claim 21, wherein producing the FDD score comprises determining summary statistics for the distribution of the fractal measurements.

23. The method of claim 22, wherein the summary statistics comprise one or more of a standard deviation of the fractal measurements, and skewness of the fractal measurements, kurtosis of the fractal measurements.

24. The method of claim 21, wherein the output comprises a continuous score indicating the probability that a subject has Alzheimer’s disease, and:
further comprising allocating the continuous score into one of a plurality of bins, wherein each bin corresponds to a subject’s risk of developing Alzheimer’s disease; and/or
wherein when the probability drops below a threshold, the output provides an indication that the subject does not have Alzheimer’s disease.

25-26. (canceled)

27. The method of claim 21, wherein the output includes an identification of the extracted measures analyzed by the machine learning system and/or the FDD scores used to provide the output, and optionally wherein the extracted measures provided in the output are identified using a pictorial representation of the analyzed measure.

28. (canceled)

29. The method of claim 21, wherein the neural data is recorded from the subject over at least one period of time during which the subject is at rest, and:
wherein the neural data is recorded from the subject during at least two periods of time;
wherein the method further comprises providing an output of the subject’s AD status after every period of time during which neural data is recorded from the subject; and/or
wherein the method further comprises combining the results from each output to produce a longitudinal assessment of the subject’s AD status.

30. The method of claim 21, further comprising annotating the recorded neural data with one or more annotations identifying one or more of the subject’s age, sex, medical history, results from one or more biomolecular assay, and/or subjective cognitive assessment, and further comprising providing the annotations to the machine learning system for analysis with the combined extracted measures.

31-35. (canceled)

36. The system of claim 21, wherein the fractal measurement is a calculated a Katz fractal dimension (KFD).

37. The system of claim 21, wherein the fractal measurement is a calculated Higuchi fractal dimension (HFD), and wherein the calculated HFD is calculated using a kmax estimated as optimal based on a duration of the at least one measured time series.

38. (canceled)
